(19) United States
(12) Patent Application Publication
(10) Pub. No.: US 2018/0140783 A1
(43) Pub. Date: May 24, 2018

(54) ACTUATED NEEDLE SHIELDING AND SHETHING DEVICE

(71) Applicant: E3D AGRICULTURAL COOPERATIVE ASSOCIATION LTD, Merom Hagalil (IL)

(72) Inventors: Lior Raday, Kibbutz Bror-Hail (IL); Ehoud Carmel, Yehud Monosson (IL); Lior Mareli, Rehovot (IL); David Daily, Herzelia (IL); Guy Keenan, Tel Aviv (IL)

(73) Assignee: E3D AGRICULTURAL COOPERATIVE ASSOCIATION LTD, Merom Hagalil (IL)

(21) Appl. No.: 15/819,223

(22) Filed: Nov. 21, 2017

Related U.S. Application Data
(60) Provisional application No. 62/425,082, filed on Nov. 22, 2016.

(51) Int. Cl. A61M 5/32

(52) U.S. Cl. CPC A61M 5/3204; A61M 5/3245; A61M 5/3257; A61M 5/3271

(57) ABSTRACT

The disclosure relates to needle shielding devices which cover injector needle to prevent accidental needle pricks and reduce user fear, before, during and following injection. More particularly, the disclosure relates to sheathed needle actuation devices configured to provide a predetermined force-distance profile on the shield during injection.

### REFERENCE TO RELATED APPLICATIONS

Reference is hereby made to U.S. Provisional Patent Application 62/425,082, filed Nov. 22, 2016 and entitled “ACTUATED NEEDLE SHIELDING AND SHETHING DEVICE”, the disclosure of which is incorporated by reference in its entirety and priority of which is hereby claimed pursuant to 37 CFR 1.78(a) (4) and (5)(i).

### BACKGROUND

The disclosure is directed to needle shielding devices which cover injector needle to prevent accidental needle pricks and reduce user fear, both before and during injection. More particularly, the disclosure is directed to sheathed needle actuation devices configured to provide a predetermined force-distance profile during injection.

Hypodermic syringes are typically used to deliver predetermined doses of liquid medicament to a patient. However, with recent increase in healthcare costs, treatment has shifted to the home resulting in many medicaments being self-administered (e.g., insulin, β-interferon, etc.). The manipulation of a hypodermic syringe necessary to carry-out an injection may be difficult, inconvenient and anxiety-filled, particularly where the injection is self-administered. Medication delivery pens or pen injectors have therefore been developed to facilitate self-administration of injections. Pen injectors may include a generally tubular body portion which is sized and shaped to receive a cartridge carrying a medicament and having a pierceable closure, such as a rubber septum, on one end and a movable stopper-provided at an opposite end and typically inside of the cartridge. A known pen needle may be removably secured to an end of the pen injector.

The pen needle typically includes a hub that carries a double-ended needle cannula and that is configured to be removably coupled to the pen injector. The needle cannula has a first end for piercing the closure of the cartridge containing the medicament when the pen needle is secured to the pen injector. The needle cannula can also be double ended with a second end having a sharpened tip for piercing the skin of a patient during use of the pen injector. The pen needle may also have a removable cap that covers the second end of the needle cannula prior to use, to address sterility.

Likewise, shield systems have also been developed for hypodermic syringes wherein a tubular shield is moved to enclose the needle cannula and optionally lock in place following injection. Such safety shield systems are typically operated manually or are biased to cause the tubular shield to enclose the needle cannula following injection. Syringes equipped with such safety shield systems are typically discarded completely (i.e., syringe and safety shield system) after use.

One problem with other pen needle accessories, such as hidden needle adapters, has been potential needle sticks to the user during assembly of the accessory on the pen injector. Because the shield must be retractable for injection and the shield and cap assembly is typically threaded on the pen needle dispenser, the natural tendency of the user or patient is to press the cap toward the injector during assembly. This may cause the needle to pierce the cap and possibly puncture the user during assembly. Another problem associated with pen needles has been the safe disposal of the hub and double ended needle cannula. It would be most desirable to safely enclose both sharp ends of the needle cannula hub assembly to avoid inadvertent punctures during and following disposal.

Accordingly, there is a need for a safety needle actuator capable of providing a desirable force-distance profile.

### SUMMARY

In an embodiment, provided is a safety shield member assembly comprising: a removable housing member having a proximal end and a distal end; a sleeve member adapted to receive and engage a proximal end of a body comprising an injectable compound, having a longitudinal axis, a proximal end and a distal end, the sleeve member defining a central axial flanged column configured to receive and engage a needle cannula having a proximal end and a distal end; a needle cannula having a proximal end and a distal end, operably coupled to the sleeve member; a shield member having a longitudinal axis, a distal end coupled to a sheath member and a proximal end defining a central aperture accommodaing the proximal end of the needle cannula; the sheath member having an open distal end and open proximal end, the sheath member being moveably slidably (and rotatably in certain embodiments) coupled to the shield member and configured to move between a first position surrounding the needle cannula and a second position exposing the needle cannula; and a biaser operably coupled to the needle shield for biasing the needle shield toward proximal end, wherein the assembly is configured to provide a predetermined profile of force on the shield as a function of distance traveled by the shield during the movement of the shield member relative to the sleeve member.

In another embodiment, the shield is exposed to a predetermined profile of force as a function of the distance traveled in mm, wherein, on initiation of movement during injection: the shield is configured to be exposed to a force of between about 2.5 N and about 3.5 N within about 0.2 mm and about 1.2 mm; between about 2.0 mm and about 9.4 mm, the shield is configured to be exposed to an increase (ΔN/mm) in force of between about 0.2 N and about −0.4 N; and between about 9.0 mm and about 11 mm, the shield is configured to be exposed to a force of between about 2.8 N and about 3.8 N.

In yet another embodiment, provided herein is an injection device comprising the partially rotating embodiment or the linear embodiment of the sheathed needle actuation devices described herein.

In still another embodiment, provided herein is a safety needle shield assembly comprising a sleeve member adapted to receive and engage a proximal end of a body comprising an injectable compound, having a longitudinal axis, a proximal end and a distal end; a needle cannula having a proximal end and a distal end, operably coupled to the sleeve member; a shield member having a longitudinal axis, a distal end coupled to a sheath member and a proximal end defining a central aperture accommodating the proximal end of the needle cannula; the sheath member having an open distal end and open proximal end, the sheath member being moveably slidably coupled to the shield member, the shield member configured to move between a first position surrounding the needle cannula and a second position exposing the needle cannula; and a biaser operably coupled to the needle shield for biasing the shield member toward proximal end, wherein the shield member, sheath member and biaser are all configured to act as a single component in the second position exposing the needle cannula.

### BRIEF DESCRIPTION OF THE DRAWINGS

The features of the sheathed needle actuation devices and their methods of use described herein will become apparent from the following detailed description when read in conjunction with the drawings, which are exemplary, not limiting, and wherein like elements are numbered alike in several figures and in which:

FIG. 1A, illustrates top plan view, side elevation view in FIG. 1B, bottom perspective view in FIG. 1C, and top perspective view in FIG. 1D of the partially rotating embodiment of the sheathed needle actuation devices described and claimed;

FIG. 2, illustrates an exploded isometric view thereof;

FIG. 3A, Illustrates an isometric view and in FIG. 3B—X-Z cross section A-A of the sleeve member in FIG. 3A;

FIG. 4A, illustrates an isometric view of the sheath member in the partially rotating embodiment of the sheathed needle actuation device, with X-Z cross section B-B illustrated in FIG. 4B;

FIG. 5A, illustrates an isometric perspective view of the shield member of the partially rotating embodiment of the sheathed needle actuation device, with FIGS. 5B, and 5C illustrating cutaway views thereof;

FIG. 6A, illustrates X-Z cross section of the partially rotating sheathed needle actuation device in stowed position, with enlarged section A illustrated in FIG. 6B, enlarged FIG. 6A in FIG. 6C and an isometric view thereof in FIG. 6D;

FIG. 7A, illustrates X-Z cross section of the partially rotating sheathed needle actuation device upon coupling to an injector (e.g., pen injector), with enlarged section B illustrated in FIG. 7B, an enlarged cutaway X-Z view of FIG. 7A in FIG. 7C, and a top isometric perspective view of the cutaway in FIG. 7D;

FIG. 8A, illustrates partial cutaway isometric cross section of the partially rotating sheathed needle actuation device upon initial actuation, with enlarged view of section C in FIG. 8B, and X-Z cross section view thereof in FIG. 8C with an enlarged section illustrated in FIG. 8D;

FIG. 9A, illustrates partial cutaway isometric view of the partially rotating sheathed needle actuation device upon completion of initial actuation, enlarged isometric view illustrated in FIG. 9B, X-Z cross section view illustrated in FIG. 9C and enlarged X-Z cross section illustrated in FIG. 9D;

FIG. 10A, illustrates isometric partial cutaway of the partially rotating sheathed needle actuation device during injection, enlarged section E illustrated in FIG. 10B, X-Z cross section elevation view illustrated in FIG. 10C, and enlarged X-Z cross section illustrated in FIG. 10D;

FIG. 11A, illustrates isometric perspective view of the partially rotating sheathed needle actuation device upon initiation of sheathing, with enlarged isometric perspective view F thereof illustrated in FIG. 11B, a partial cutaway isometric cross section view thereof illustrated in FIG. 11C, and enlarged isometric perspective view of a section illustrated in FIG. 11D, 11E illustrating X-Z cross section elevation view of the partially rotating sheathed needle actuation device upon initiation of sheathing, and enlarged portion thereof in FIG. 11F;

FIG. 12A, illustrates isometric perspective view of the partially rotating sheathed needle actuation device upon completion of needle sheathing, with X-Z cross section elevation view illustrated in FIG. 12B and enlarged X-Z cross section elevation view illustrated in FIG. 12C;

FIG. 13, illustrates a comparison between the sheathed needle actuation devices described herein and currently available sheathed needle actuation devices;

FIG. 14A, illustrates illustrates top plan view, side elevation view illustrated in FIG. 14B, bottom perspective view illustrated in FIG. 14C, and top perspective view illustrated in FIG. 14D of the linear motion embodiment of the sheathed needle actuation devices;

FIG. 15, illustrates an exploded isometric view thereof;

FIG. 16A illustrates an isometric view of sleeve member in FIGS. 15 and 16B illustrates X-Z cross section B-B elevation view of the sleeve member in FIG. 15;

FIG. 17A illustrates an isometric view of the sheath member of linear motion embodiment of the sheathed needle actuation device and FIG. 17B illustrates X-Z cross section B-B elevation view thereof;

FIG. 18A illustrates an isometric view of the shield member of the linear motion embodiment of the sheathed needle actuation device and FIG. 18B illustrates partial cutaway view thereof;

FIG. 19A, illustrates X-Z cross section elevation view of the linear motion sheathed needle actuation device in stowed position and enlarged section H illustrated in FIG. 19B;

FIG. 20A, illustrates X-Z cross section elevation view of the linear motion sheathed needle actuation device upon partial actuation and enlarged section I thereof illustrated in FIG. 20B;

FIG. 21A, illustrates X-Z cross section elevation view of the linear motion sheathed needle actuation device during injection and enlarged section J thereof, illustrated in FIG. 21B;

FIG. 22A, illustrates X-Z cross section elevation view of the linear motion sheathed needle actuation device upon completion of initial actuation and enlarged section K thereof, illustrated in FIG. 22B;

FIG. 23A, illustrates X-Z cross section elevation view of the linear motion sheathed needle actuation device upon completion of needle sheathing and enlarged section L thereof, illustrated in FIG. 12B; and

FIG. 24, illustrates the force distance profile of the sheathed needle actuation device.

While the disclosure is amenable to various modifications and alternative forms, specifics thereof have been shown by way of example in the drawings and will be further described in detail hereinbelow. It should be understood, however, that the intention is not to limit the disclosure to the particular embodiments described. On the contrary, the intention is to cover all modifications, equivalents, and alternatives.

### DETAILED DESCRIPTION

The disclosure relates in one embodiment to shielding devices which cover injector needle to prevent accidental needle pricks and reduce user fear, both before and during injection. In another embodiment, the disclosure relates to sheathed needle actuation devices configured to provide a predetermined force-distance profile during injection. The shielding device can be integral to the injection device or as an add on, to be coupled to the injection device by the user or a care giver/physician.

Detailed embodiments of the present technology are disclosed herein; however, it is to be understood that the disclosed embodiments are merely exemplary, which can be embodied in various forms. Therefore, specific structural and functional details disclosed herein are not to be interpreted as limiting but merely as a basis for the claims and as a representative basis for teaching one skilled in the art to variously employ the present technology in virtually any appropriately detailed structure. Further, the terms and phrases used herein are not intended to be limiting but rather to provide an understandable and enabling description.

The terms “first,” “second,” and the like, herein do not denote any order, quantity, or importance, but rather are used to denote one element from another. The terms “a”, “an” and “the” herein do not denote a limitation of quantity, and are to be construed to cover both the singular and the plural, unless otherwise indicated herein or clearly contradicted by context. The suffix “(s)” as used herein is intended to include both the singular and the plural of the term that it modifies, thereby including one or more of that term (e.g., the groove(s) includes one or more groove). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

In addition, for the purposes of the present disclosure, directional or positional terms such as “top”, “bottom”, “upper,” “lower,” “side,” “front,” “frontal,” “forward,” “rear,” “rearward,” “back,” “trailing,” “above,” “below,” “left,” “right,” “radial,” “vertical,” “upward,” “downward,” “outer,” “inner,” “exterior,” “interior,” “intermediate,” etc., are merely used for convenience in describing the various embodiments of the present disclosure.

In an embodiment, provided herein is a a safety shield member assembly comprising: a housing member having a proximal end and a distal end, adapted to receive and engage a proximal end of a body comprising an injectable compound; a sleeve member, having a longitudinal axis, a proximal end and a distal end, the sleeve member defining a central axial fluted tubular portion configured to receive and engage a needle cannula having a proximal end and a distal end; a needle cannula having a proximal end and a distal end, operably coupled to sleeve member; a shield member having a longitudinal axis, a distal end slidably coupled to the central fluted tubular portion of the sleeve member and a proximal end defining a central aperture accommodating the proximal end of the needle cannula; a sheath member having an open distal end and open proximal end, the sheath member being moveably slidably coupled to the shield member and configured to move between a first position surrounding the needle cannula and a second position exposing the needle cannula; and a biaser operably coupled to the shield member for biasing the shield member toward proximal end, wherein the assembly is configured to provide a predetermined profile of force on the shield as a function of distance traveled by the shield during the movement of the sheath. The term “accommodating” and its grammatical derivations refers, for example to being configured to allow the needle cannula to traverse through.

The term “biaser” refers in an embodiment to any component that is provided for exerting a force on another component or element and/or components or elements to ensure that the component and/or components are forced together (e.g. into engagement) or forced apart (e.g. out of engagement). The biaser may be manufactured from any suitable flexible energy storage material known by a person skilled in the art (e.g. metal, rubber or plastics) and may take any suitable form, e.g., a spring. The biaser can be provided as “armed” or in other words, the energy is already stored and under the proper circumstances, biasing will cause the energy to be released in the component(s) or element(s) on which the biaser acts, will be forced to engage or disengage.

In general, the shielding device provided comprises a hub, the hub nesting a sheath and a shield, where through reciprocating movement during injection, for example with a pen injector, a needle cannula coupled on the hub is exposed to a predetermined length while still being concealed from the user, by using the injection site as counter surface affecting the movement of the nested components, is translated distally, reaches an end point, and upon retracting the needle from the injection site, separating the sheath from the shield locking the shield around the needle in such a way that reuse of the needle is impracticable, needle prick of spent needle is highly unlikely and the needle remains concealed at all times from the user. Accordinly, after the housing covering the shielding device is removed. The steps involved in exposing the needle, penetrating the injection site, injecting the entire medication in the injector, and removing the injector from the injection site are mirrored in the force profile borne by the shield during the process, as a function of the distasnce “traveled” by the shield in the recioprocating motion,

The shielding devices which cover injector needle to prevent accidental needle pricks and reduce user fear, both before and during injection can operate in a predetermined sequence of operations, whereby a needle cannula, open on both sides is partially exposed, can be coupled to a proximal end (the end usually closer to the patient in operation) of the injector and be configured to penetrate a septum or similar barrier. A shield coupled initially to a sheath, around the needle cannula, can be movable between a first position covering the needle to a second position exposing the needle cannula. Typically an actuation step (in other words, the user-related input responsible for both energizing and release of the shield) involves application of force on the shield for either arming the biaser operably coupled to the shield and/or the sheath and subsequent abrupt urging of the shieth and/or shield either distally (the sheath) or proximally (the shield).

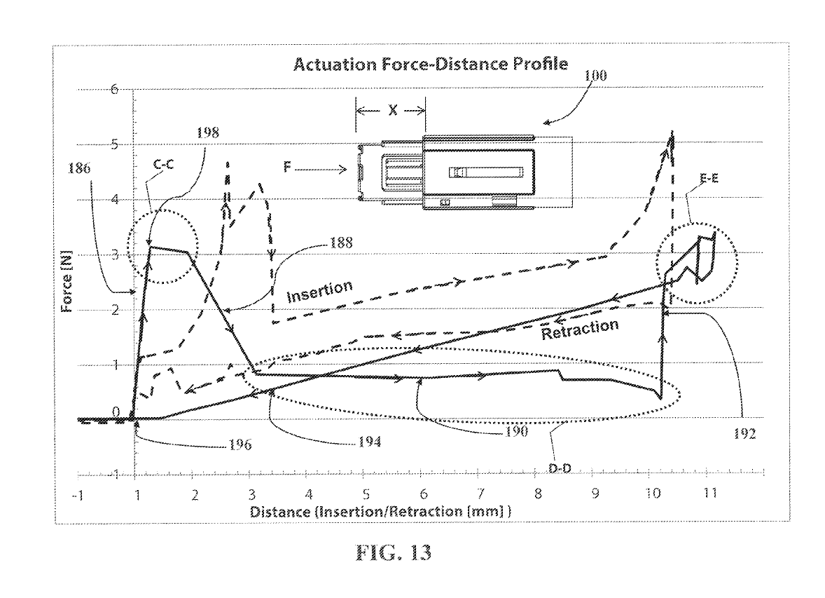

It has been found, that higher arming forces (see e.g, FIG. 13, peak force C-C), creates a reflexive (i.e. involuntary) recoil by the user, which may result in the user removing the injector before the full dose of medication has been delivered to the injection site.

Further, during the motion of the shielding member during the injection, a second arming mechanism is employed to deploy a sheth to lock and cover the needle cannula in the protracted position, to prevent reuse of the device and prevent accidental needle prick. An increase in the force on the shield member necessary to arm the sheath that is higher than a given threshold was found to induce users to reduce the pressure on the injector, thereby creating uncertainty as to both the amount of injectable medication delivered, as well as repeatability between injections. Accordingly, essentially a substantially flat profile of force in Newtons (N) as a function of the distance “travelled” by the shield during the sequence of operation (see e.g., FIG. 13, portion D-D), can improve repeatability and reduce the uncertainty.

Finally, during the arming of the sheath member at the end of injection and the recovery of the shield to its shielding position, another peak in force is observed. Again, too high force at that region can create circumstances where the user does not cover the needle cannula completely or consistently, which may result in the sheath not locking in place, leaving the needle cannula exposed.

Accordingly and in an embodiment, the shield can be configured by the mechanism described herein to be exposed to a predetermined profile of force as a function of the distance traveled in millimeters (mm), wherein, on initiation of movement during injection: the shield is configured to be exposed to a force of between about 2.5 N and about 3.5 N within about 0.2 mm and about 1.2 mm; between about 2.0 mm and about 9.4 mm, the shield is configured to be exposed to an increase (αN/mm) in force of between about 0.2 N and about −0.4 N; and between about 9.0 mm and about 11 mm, the shield is configured to be exposed to a force of between about 2.8 N and about 3.8 N.

The sheathed needle actuation devices configured to provide a predetermined force-distance profile during injection, can be enclosed in a hermetically sealed housing, that is open on one end (the distal end), and be sealed with a peelable reed or tab. In an embodiment, the term “peelable” refers to securing in an impervious manner by adhesive bonding or sealing, enabling the manual separation, in normal use of the reed or tab, be it by means of an adhesive, heat sealing, scoring, or other means, can be broken, disrupted or eliminated by manually urging the locator strip away from the upper film without compromising the integrity of the films.

The term “coupled”, including its various forms such as “operably coupled”, “coupling” or “coupleable”, refers to and comprises any direct or indirect, structural coupling, connection or attachment, or adaptation or capability for such a direct or indirect structural or operational coupling, connection or attachment, including integrally formed components and components which are coupled via or through another component or by the forming process (e.g., an electromagnetic field). Indirect coupling may involve coupling through an intermediary member or adhesive, or abutting and otherwise resting against, whether frictionally (e.g., against a housing) or by separate means without any physical connection.

A more complete understanding of the components, processes, assemblies, and devices disclosed herein can be obtained by reference to the accompanying drawings. These figures (also referred to herein as “FIG.”) are merely schematic representations (e.g., illustrations) based on convenience and the ease of demonstrating the present disclosure, and are, therefore, not intended to indicate relative size and dimensions of the devices or components thereof and/or to define or limit the scope of the exemplary embodiments. Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

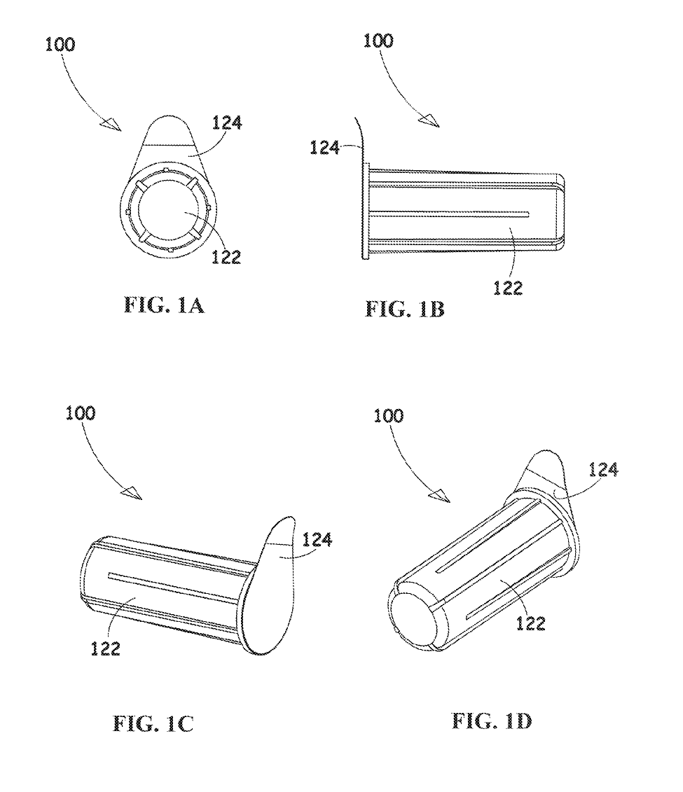

Turning now to FIG. 1, illustrating top (1A) plan view, side (1B) elevation view, bottom perspective view (1C), and top perspective view (1D) of the partially rotating embodiment of the sheathed needle actuation device described and claimed. As illustrated, sheathed needle actuation device 100 configured to provide a predetermined force-distance profile during injection, can be enclosed in housing 122, having an open distal end sealed with a peelable cover tab 124. Shielding device can be formed of biocompatible polymer and be provided either as a separate assembly from the pen injector, or in an embodiment, already coupled to the injector.

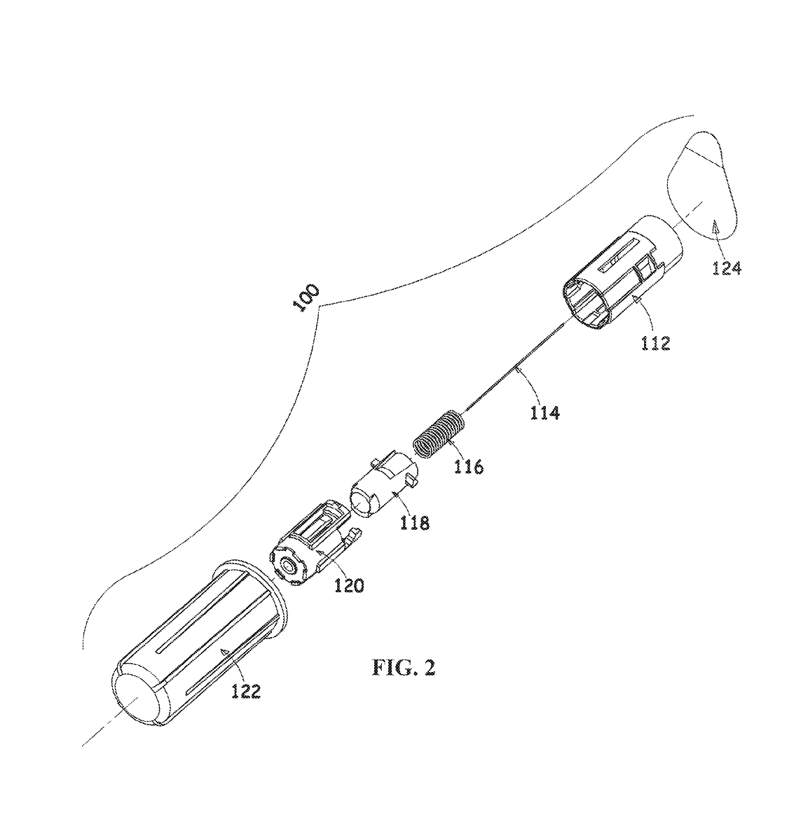

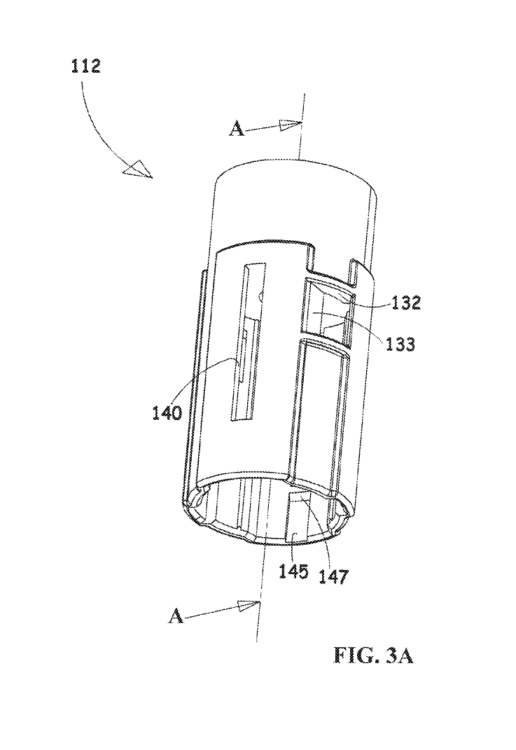

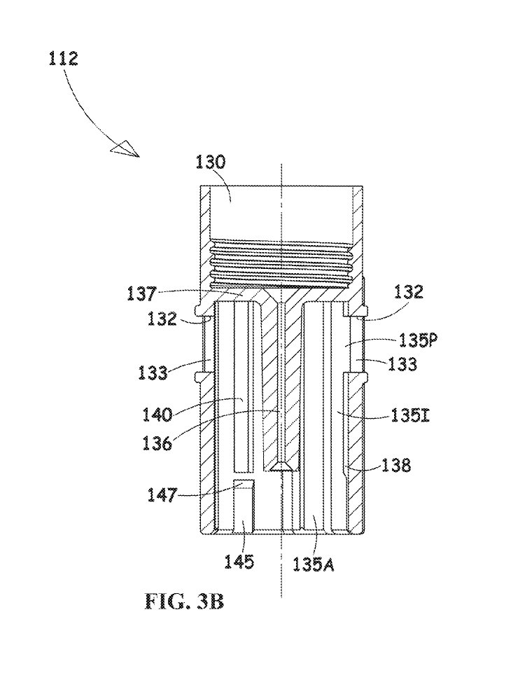

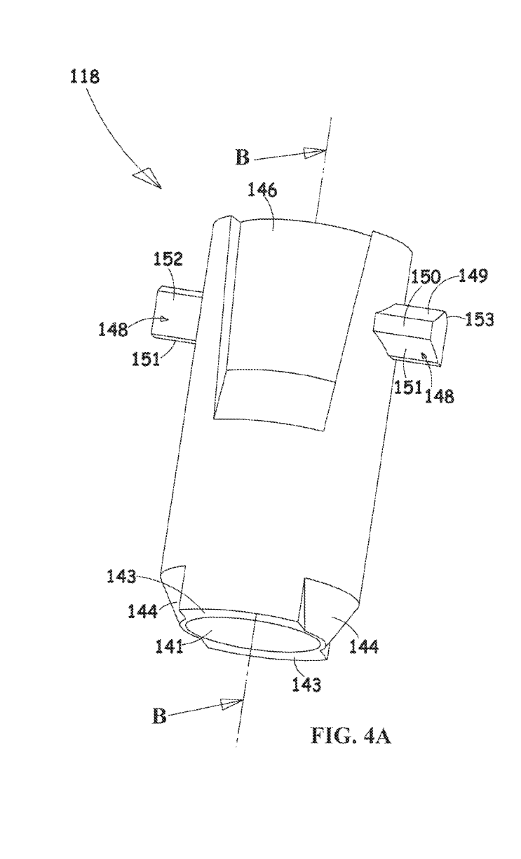

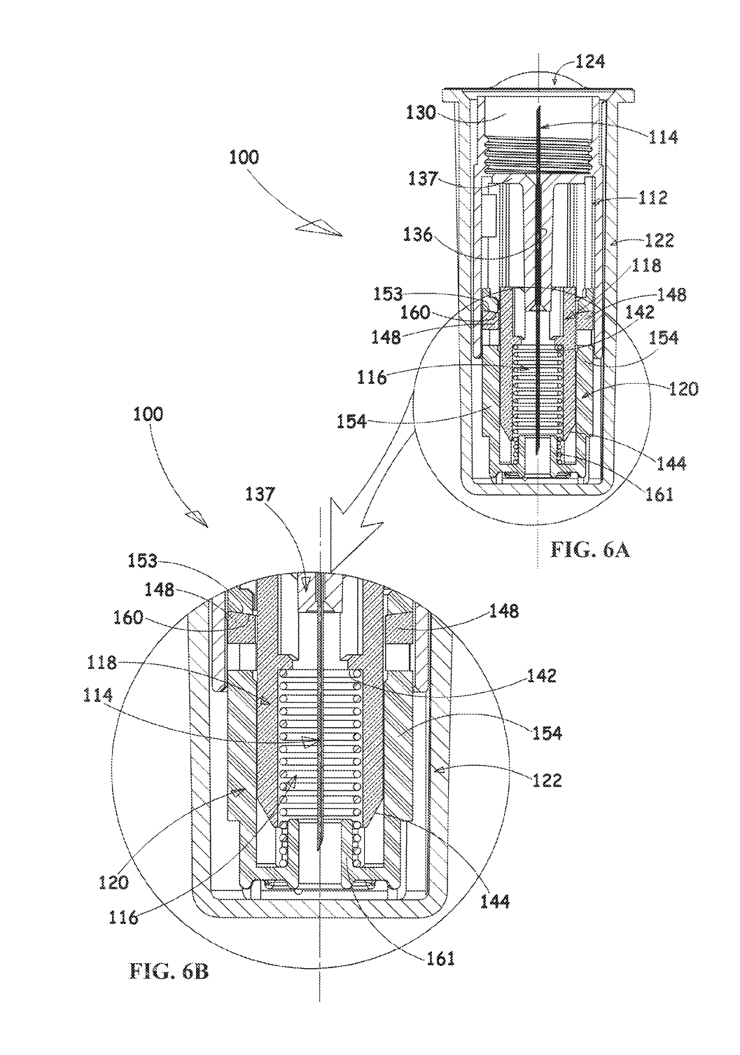

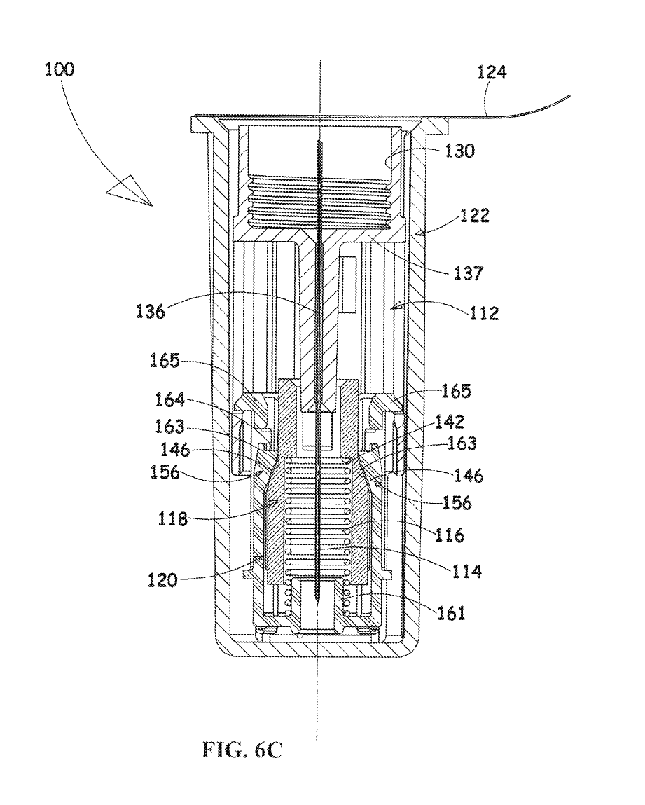

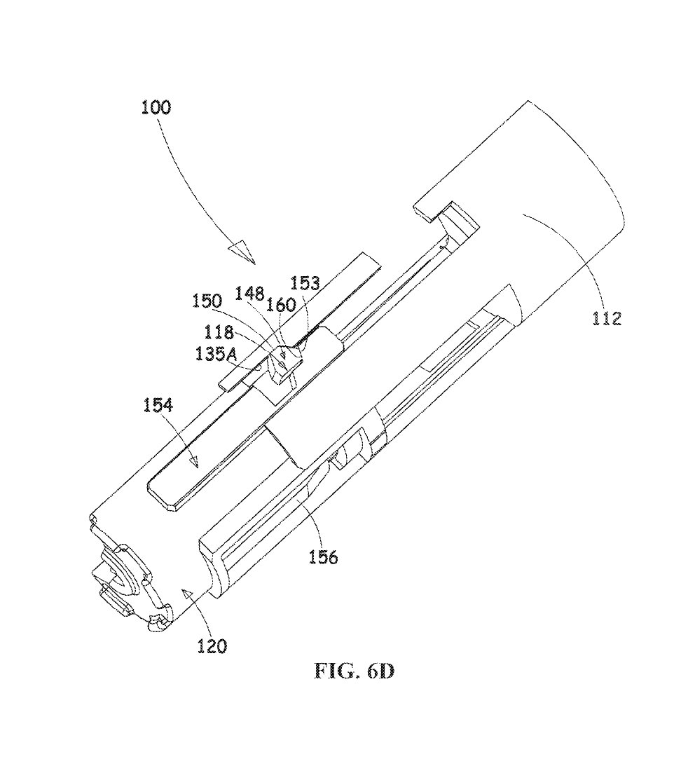

Turning now to FIGS. 2-5, illustrating in FIG. 2 an exploded isometric view of the partially rotating embodiment of the sheathed needle actuation device 100 described herein, wherein device 100 can comprise housing member 122 having a longitudinal axis, proximal end and a distal end. Sleeve member 112 can be adapted to receive and engage a proximal end of a body (for example, an autoinjection pen) comprising an injectable compound (not shown, see e.g., FIG. 6). Sleeve 112 can have a longitudinal axis, a proximal end and a distal end and define central axial flanged column 137 (See e.g., FIG. 3), which can be configured to receive and engage needle cannula 114 having a proximal end and a distal end. Needle cannula 114 can have a proximal end and a distal end, and be operably coupled to sleeve member 112. Also shown is shield member 120 having a longitudinal axis, a distal end that can be slidably coupled to central flanged column 137 (see e.g., FIG. 3) of sleeve member 112 and a proximal end defining a central aperture accommodating the proximal end of needle cannula 114. FIG. 2 shows sheath member 118 having an open distal end and open proximal end. Sheath member 118 can be moveably slidably coupled to shield member 120 and be configured to move between a first position surrounding needle cannula 114 and a second position exposing needle cannula 114. Also illustrated in FIG. 2 is biaser 116 operably coupled to shield member 120 for biasing shield member 120 toward proximal end at the end of the process, wherein the assembly is configured to provide a predetermined profile (see e.g., FIG. 13) of force on shield member 120 as a function of distance traveled by shield member 120 during injection.

One or more components may be referred to herein as “configured to,” “configured by,” “configurable to,” “operable/operative to,” “adapted/adaptable,” “able to,” “conformable/conformed to,” etc. The terms (e.g. “configured to”) can generally encompass active-state components and/or inactive-state components and/or standby-state components, unless context requires otherwise. Also, the term “slidably coupled”, or “slidably” can be used in its broadest sense to refer to elements which are coupled in a way enabling one element to slide or translate with respect to another element.

Turning now to FIG. 3A, 3B, illustrating an isometric view of sleeve member 112 (FIG. 3A) and X-Z cross section A-A of the sleeve member in FIG. 3A in FIG. 3B. As illustrated, sleeve member 112 of partially rotating embodiment of the sheathed needle actuation device 100 can have, (for example, a medical pen injector) an injector engaging portion 130 (see e.g., FIG. 3B) disposed on the distal end of sleeve 112. As illustrated in FIG. 3A, sleeve member 112 can comprise radial, quadrilateral distal openings 132 with radial beveled facets 133. Also illustrated are guiding slots 140. FIGS. 3A & 3B also illustrate recessed portion 145 and recess frame ridge 147.

X-Z Cross section A-A in FIG. 3B illustrates flanged needle column 137, having needle bore 136, configured to receive and engage needle cannula 114. Sleeve member 112 also has at least pair of quadrilateral distal opening 132 disposed radially above the flanged portion of needle column 137, each quadrilateral opening 132 has a pair of parallel vertical facet and a pair of parallel radial facets, and wherein the radial facets disposed closer to the distal end of sleeve member 112 are beveled facets 133. Internal walls of sleeve member 112 can also define at least a pair of axially disposed groove(s), each having anterior channel portion 135A, intermediate portion 135I and posterior portion 135P, wherein abutment 138 extends along the entire length of intermediate portion 135I. as well as at least a pair of shield member guiding slots 140. It is to be understood that the term “abutment” is being used in an embodiment, to refer to the end structures against which other elements or members can slidably translate or press.

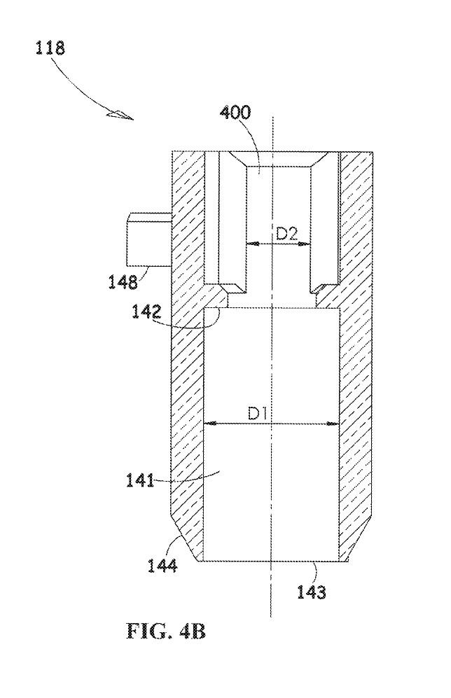

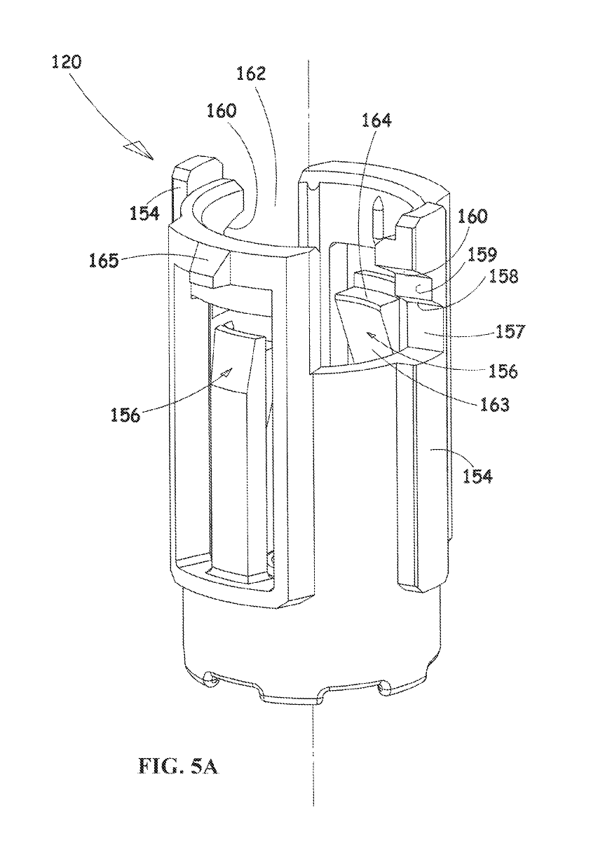

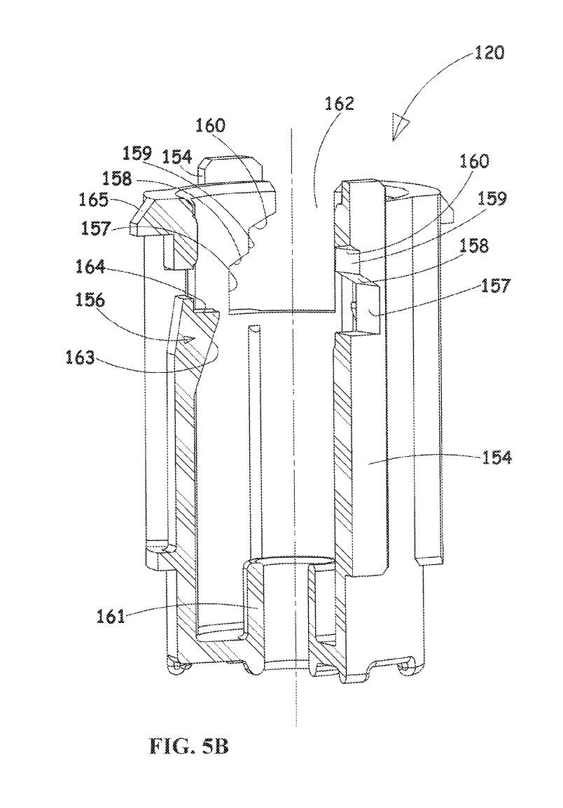

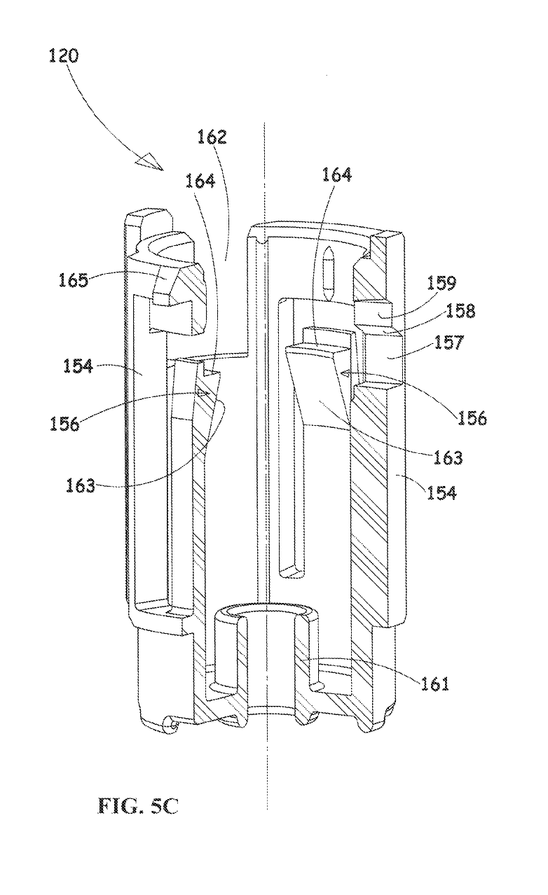

Turning now to FIG. 4, illustrating in FIG. 4A an embodiment of sheath member 118 of partially rotating sheathed needle actuation device 100. As shown, sheath member 118 can comprise beveled proximal end 144 radially disposed around opening 141 at the proximal end of sheath member 118 separated by flush portion 143, such that a quarter turn of sheath member in any direction will transfer between beveled portion 144 and flush portion 143 of the proximal end of sheath member 118, with at least a pair of slanted recessed portion(s) 146 configured to receive at least a distal portion of a pair of locking arms 156 (see e.g., FIG. 5A, 5B). Sheath member additionally comprises at least a pair of shelves 148, each shelf 148 comprising front facet 150, upper dovetail facet 151, lower dovetail facet 149, plane back facet 152 and chamfered facet 153. X-Z cross section B-B of sheat member shown in FIG. 4A is illustrated in FIG. 4B. As shown, internal volume of sheath member 118 defines coaxially disposed cylinders having internal diameter D₁, configured to accommodate biaser 116, for example c spring coil having diameter that is smaller than D₁. Sheath member 118 distal end 400 (FIG. 4B) defines an internal coaxial cylinder having internal diameter D₂, configured to accommodate needle cannula 114 and an outer diameter that is equal to D₁, thereby defining rim 142 against which biaser 116 can abut in compressed position. As illustrated in FIG. 4B, distal end 400 defines a flueted bore with a diameter D2, that is configured to accommodate and slidably couple to flanged needle column 137 coaxially disposed on sleeve 112.

Turning now to FIGS. 5A and 5B, showing a cutaway illustration of shield member 120 of partially rotating embodiment of the sheathed needle actuation device 100. As illustrated, shield member 120 can comprise at least a pair of rails 154 configured to be received in sleeve member 112 guiding slots 140, wherein each of rails 154 can further define, in combination with the distal end of shield member 120 a graded recess in the circumference of shield member 120. The recess extending proximally from the distal end of the recess by first axially parallel facet 157, followed by first slanted facet 158, followed by second axially parallel facet 159, and followed by second slanted facet 160, culminating in gap 162 (see e.g., FIG. 5C), wherein gap 162 is configured to receive and frictionally engage the width of dovetail facet 151 (see e.g., FIG. 4A), in each of shelf 148. Shield member 120 can also have at least a pair of resilient locking arms 156, disposed at a 90 degree radially to rails 154. Each resilient locking arm terminating at the distal end with centrally expanding slope 163 and ledge 164 (see e.g., FIG. 5C). Slope 163 engages recess 146 of sheath member 118 while ledge 164 locks shield member 120 against flush end 143 of sheath member 118 at the end of the actuation. Also illustrated is concentric flanged ring member 161, configured to engage biaser 116 extending distally from the proximal end of shield member 120. The term “resilient” refers in an embodiment to used to qualify such flexible features as generally returning to an initial general shape without permanent deformation in element(s), e.g., resilient locking arms 156, that are provided for exerting a force on a component (e.g., sheath member 118) and/or components to ensure that these components are forced together, e.g. into engagement, or forced apart, e.g. out of engagement.

Turning now to FIGS. 6A-D, illustrating a cross-sectional view (FIG. 6A) of the sheathed needle actuation device 100 as shown in FIG. 2, an enlarged view (6B) thereof and an isometric enlargement showing partially the sheathed needle actuation device 100 in an inactive configuration. As illustrated in FIG. 6A, members of (e.g. partially rotating embodiment) of the sheathed needle actuation device 100 can be enclosed within removable housing 122 and hermetically sealed by peelable cover reed or tab 124. Biaser 116 is operably coupled to flanged column 137 and compressed (or armed) between shield member 120 and sheath member 118 against rim 142 (FIG. 6B). As illustrated, resilient locking arms 156 of shield member 120 are configured to engage recesses 146 of sheath member 118 (see e.g., FIG. 4A), and are axially aligned with beveled portion 144 of sheath member's 118 prroximal end. Shelves 148 of sheath member 118 (see e.g., FIG. 6C) are disposed in the graded recess in rails 154 of shield member 120, in such a manner that front facet 150 of sheath member are urged against anterior portion 135A of groove in sleeve member 112—(see e.g., FIGS. 6C, 3A) of sleeve 112. As illustrated for example, in FIGS. 6A-D, the front portion of rails 154 having shelves 148 of sheath member 118 disposed therein, in the aforementioned manner, occupy anterior portion 135A of groove in sleeve member 112, while the respective pair of chamfered facet 153 of sheath member 118 can be urged against second slanted facet 160 of shield member 120 by biaser 116, preventing rotation of sheath member 118 (see e.g., FIG. 6D).

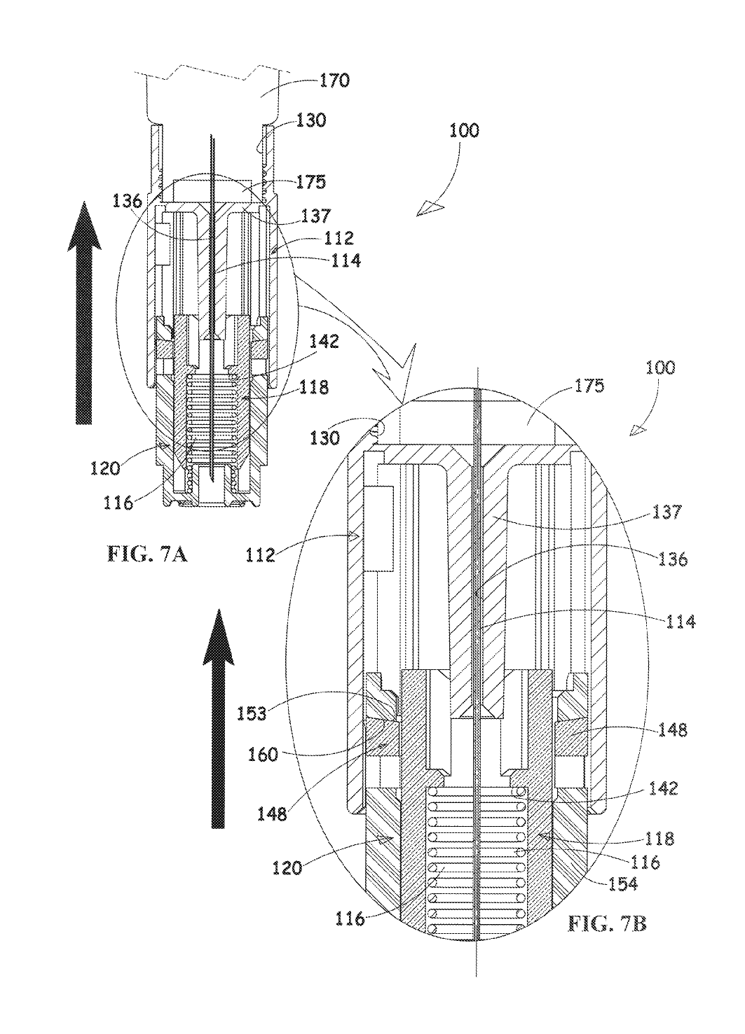

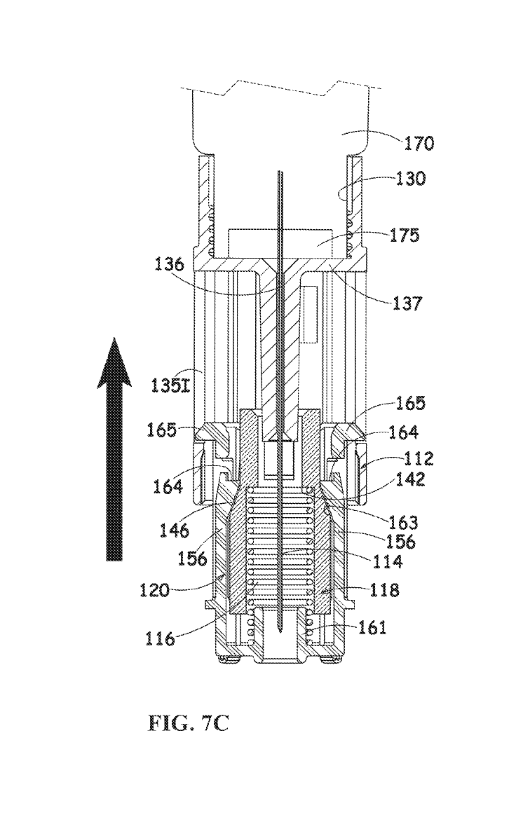

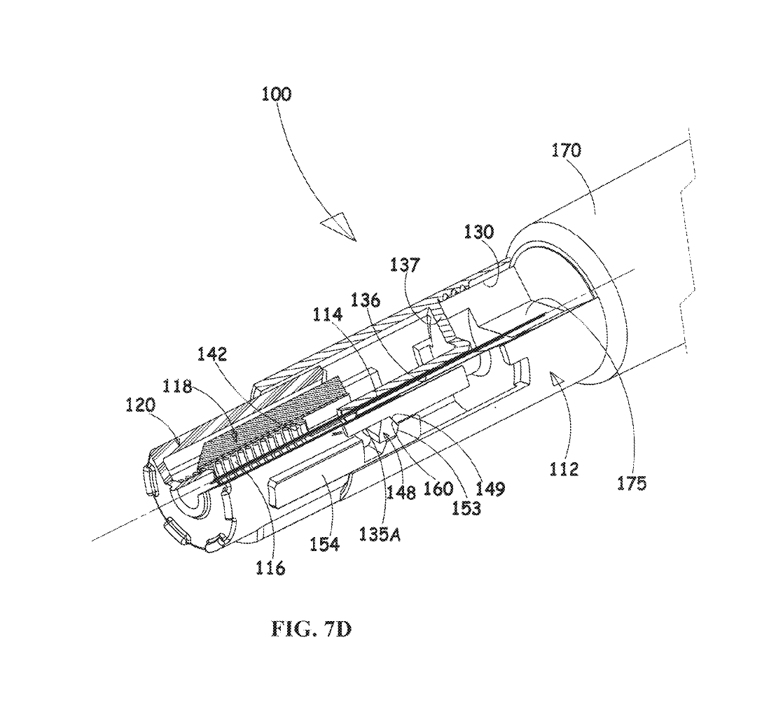

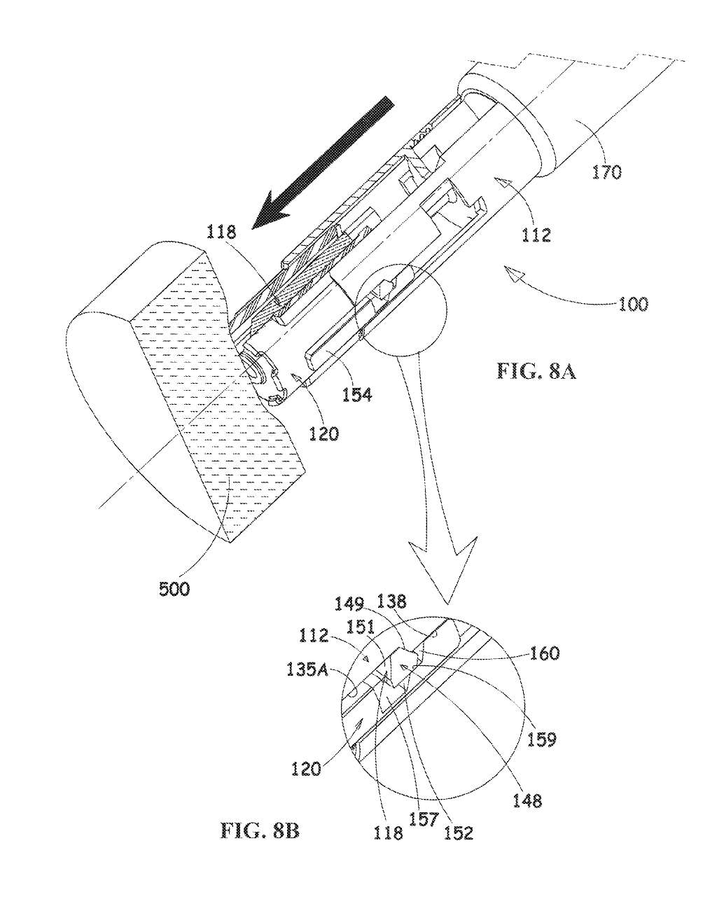

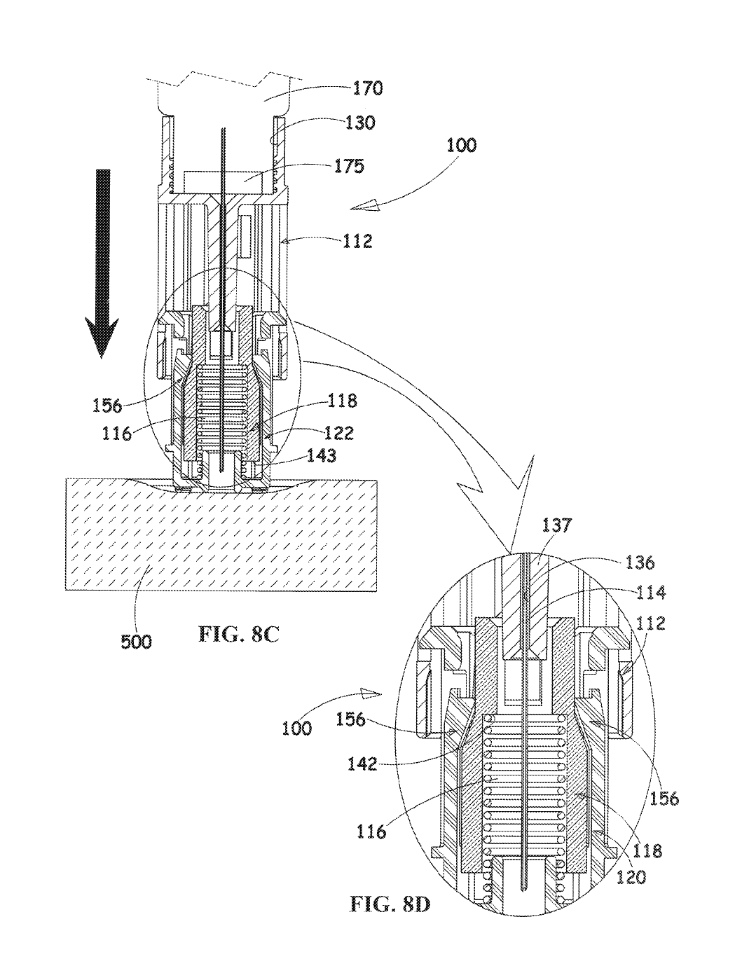

Turning now to FIG. 7, illustrating a cross section of a partially rotating embodiment of sheathed needle actuation device 100 upon coupling to an injector (e.g., pen injector) (7A) enlarged section B (7B), and enlarged isometric view in FIG. 7C. As illustrated, the user can peel cover reed or tab 124 (see e.g., FIG. 1) and engages, for example, medical pen injector 170 to injector engaging portion 130 of sleeve member 112 (see e.g., FIG. 3B), so that the distal end of needle cannula 114 penetrates into septum 175 thereof. Housing 122 can then be removed, exposing shield member 120, while needle cannula 114 remains concealed within shield member 120. As illustrated in FIG. 7B, the initial state of biaser 116 is compressed between shield member 120 and sheath member 118 against rim 142 (see e.g., FIG. 8D).

As illustrated in FIG. 7C, upper dovetail facet 149 of shelf 148 abuts the tapered edge of anterior portion 135A of sleeve member 112 such that an intial force can be exerted to start motion of sheath 118 and shield member 120 relative to sleeve member 112. The angle defined between upper dovetail facet 149 and sheath 118 longitudinal axis, can be used to determine the initial peak force threshold, the surpassing of which will cause the user to fully insert needle cannula 114 (actuate the injection, see e.g., FIG. 7D) at injection site 500.

Turning now to FIG. 8, illustrating cross section of the partially rotating sheathed needle actuation device 100 upon initial actuation (8A) enlarged section C (8B), cross ection X-Z thereof in FIG. 8C, and enlarged section of the cross section in FIG. 8D. As illustrated, in order to perform the initial actuation of partially rotating embodiment of the sheathed needle actuation device 100, the user can compress shield member 120 against injection site 500 (see e.g., FIG. 8C); whereby force applied to shield member 120 can cause relative movement between shield member 120, sleeve member 112 and sheath member 118 (see e.g., FIG. 8D). At this stage, shield member 120, sheath member 118 and biaser 116 are all configured to act as a single or in other words, a monolithic component. As illustrated in FIGS. 8B-D, shelves 148 of sheath member 118 can affect a quarter turn rotation, for example, in a clockwise direction relative to sleeve member 112, as the respective pair of upper dovetail facets 149 (see e.g., FIG. 8B) of sheath member 118 are forced against the tapered edge of anterior portion 135A of sleeve member 112 , such that plane back facet 152 of sheath member 118 can engage second axially parallel facet 159 of shield member 120, onto second slanted facet 160 and into gap 162. (See e.g., phase 186 in FIG. 13). At this point, resilient arms 156 (see e.g., FIG. 5A) can be configured to be aligned with flush portion 143 (see e.g., FIG. 4A) of sheath member 118. Again, forcing upper dovetail facet 149 (see e.g., FIG. 4A) against the tapered edge of anterior portion 135A (see e.g., FIG. 8B) of sleeve member 112 beyond the threshold that can be modulated by the proper selection of the angle of upper dovetail fact 149, will cause the user to insert needle cannula 114 at the injection site. Accordingly, the threshold necessary may increase with lower gauge needle cannula (in other words, changing from 27 gauge to 23 gauge needle cannula).

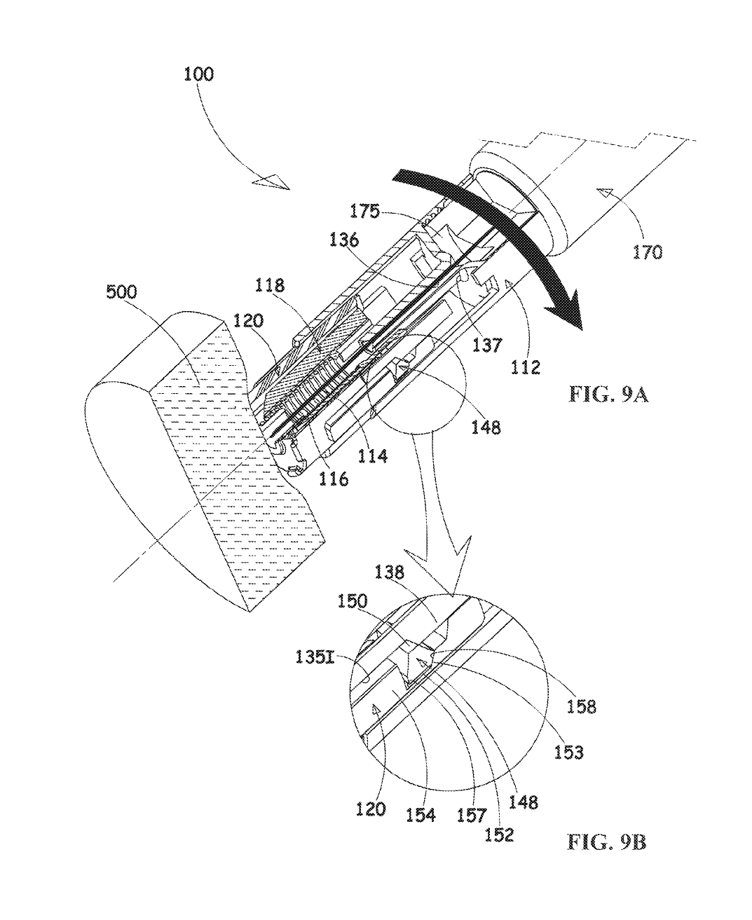

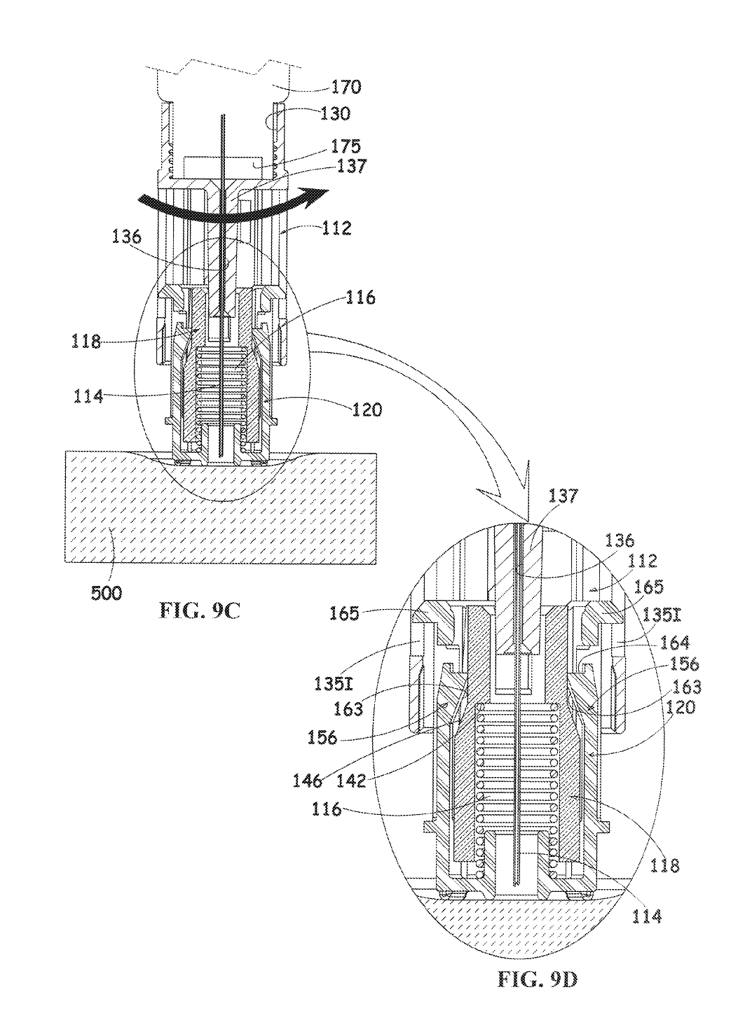

Likewise, FIG. 9 illustrating cross section of the partially rotating sheathed needle actuation device 100 upon completion of initial actuation (9A) enlarged section D (9B) and enlarged radial isometric view thereof (9C), shows how in addition to movement of shield member 120 relative to sleeve member 112 and the partial, e.g., clockwise rotation of shelves 148 of sheath member 118 as described, shelves 148 can advance into the graded recesses in rails 154, in such a manner that plane back facet 152 of sheath member 118 can abut first axially parallel facet 157 of shield member 120 and chamfered facet 153 of sheath member 118 can be urged against first slanted facet 158 of shield member 120 by biaser 116 resulting in counter-clockwise torque of shelves 148 of sheath member 118.

Due to the quarter turn rotation, the combination of shelves 148 of sheath member 118 and the front portions 165 of rails 154 (see e.g., FIG. 6C) of shield member 120 can be introduced into intermediate portion 135_{I} of sleeve member 112, so that front facet 150 of shelves 148 (see e.g., FIG. 9C) contiguously slidably translate along the longitudinal face of abutment 138 of sleeve member 112 (see e.g., FIG. 9B), resulting in chamfered facet 153 abuting recess frame ridge 147 (see e.g., FIG. 6A) and the relative locking of sheath member 118 and shield member 120.

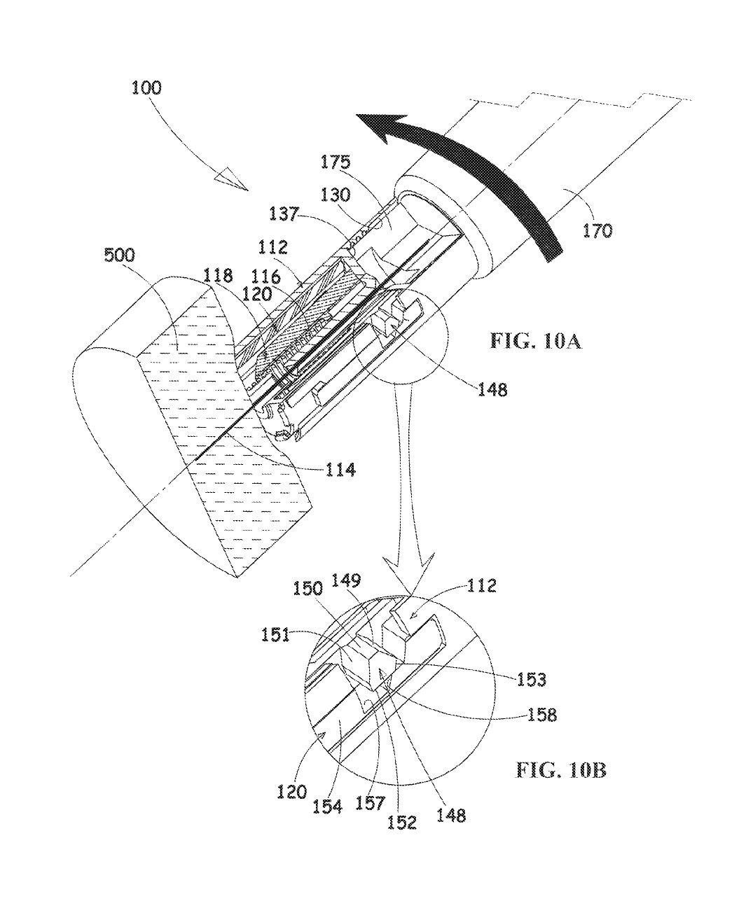

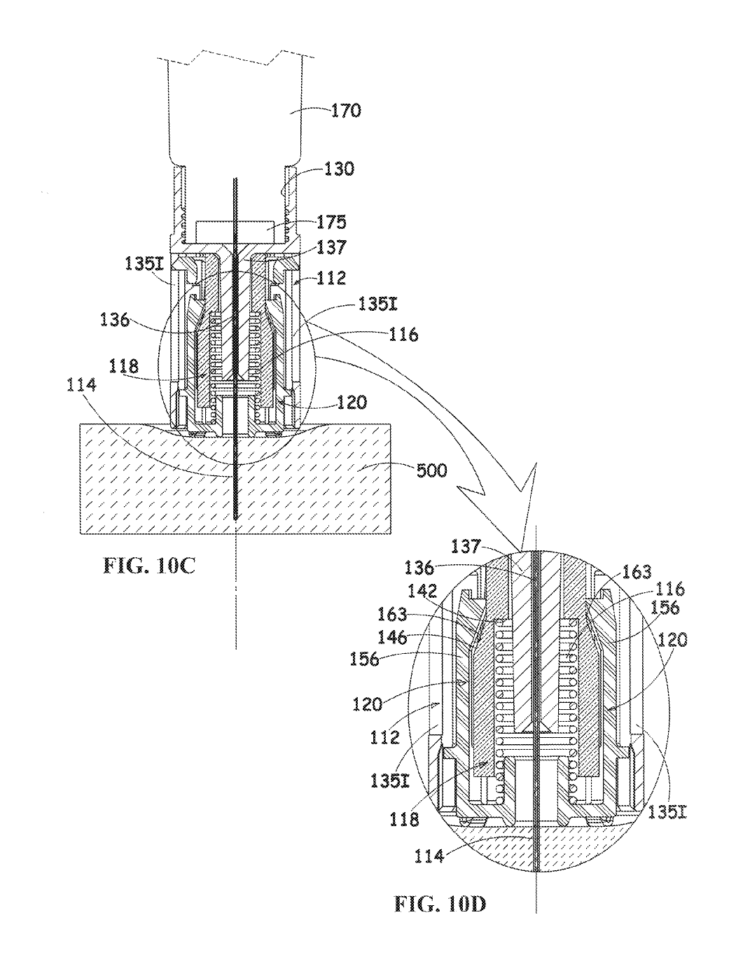

Turning now to FIGS. 10A-D, showing a cross section of the partially rotating sheathed needle actuation device 100 during injection (10A, 10C) and enlarged section E (10B). As illustrated in FIGS. 10A, 10B, to fully actuate partially rotating embodiment of the sheathed needle actuation device 100, the combination of shelves 148 of sheath member 118 and front portion 165 of rails 154 (see e.g., FIG. 6C) are advanced within intermediate portion 1351 of the grooves of sleeve member 112 (see e.g., FIG. 10D) while needle cannula 114 penetrates the injection site (see e.g., FIG. 10C), until, the combination of shelves 148 of sheath member 118 and front portion 165 of rails 154 advances distally and reaches posterior portion 135P of sleeve member 112 (see e.g., FIG. 3B) and needle cannula 114 had completed the penetration (See e.g., portion C-C, FIG. 13). Upon actuation completion of needle cannula 114 and the combination of shelves 148 of sheath member 118 and front portion 165 of rails 154 reached posterior portion 135P of the grooves of sleeve member 112, front facet 150 of shelves 148 of sheath member 118 (see e.g., FIG. 10B) no longer abuts abuttment 138 (see e.g., FIG. 9B) and shelves 148 can advance into posterior quadrilateral opening(s) 132 (see e.g., FIG. 13, portion D-D).

As shown in FIG. 9B, Upon engagement of shelves 148 in quadrilateral opening(s) 132 of sleeve member 112, shelves 148 of sheath member 118 can be separated from rails 154 of shield member 120 causing sheath member 118 to perform a quarter turn in, for example, a counter-clockwise direction relative to sleeve member 112 (see e.g., FIG. 9C) and shield member 120 and engage sleeve member 112, as chamfered facet 153 of shelves 148 of sheath member 118 can be urged in e.g., a counter-clockwise direction by first slanted facet 158 and second slanted facet 160 of shield member 120. As shelves 148 of sheath member 118 are no longer in a position to engage shield member 120, shield member 120 is free to move proximally from sheath member 118 by biaser 116 (see e.g., FIG. 9C). The quarter turn now results in ledge 164 of resilient arms 156, aligned with flush portion 143 of sheath member 118. (See e.g., FIG. 9D)

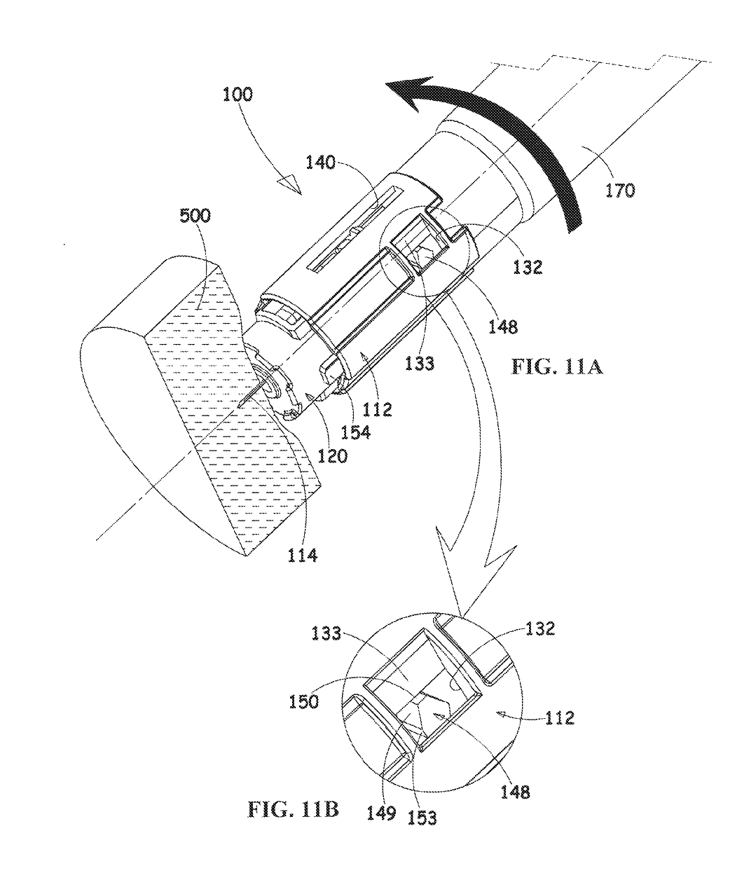

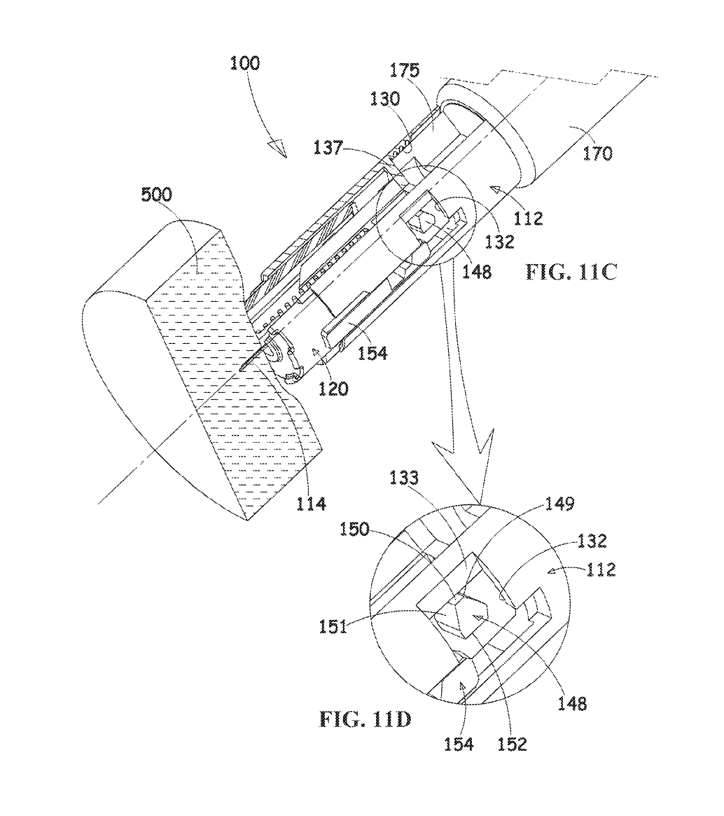

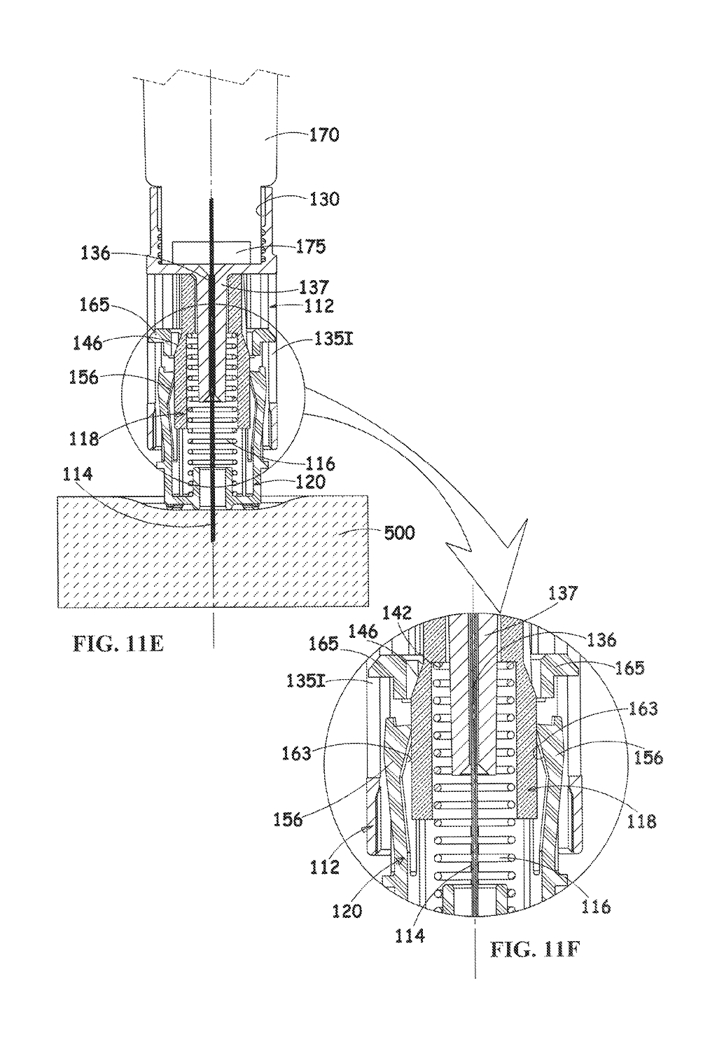

Turning now to FIGS. 11A-F, illustrating isometric perspective view of the partially rotating sheathed needle actuation device upon initiation of sheathing in FIG. 11A, with enlarged isometric perspective view F thereof illustrated in FIG. 11B, a partial cutaway isometric cross section view thereof illustrated in FIG. 11C, and enlarged isometric perspective view of a section illustrated in FIG. 11D, 11E illustrating X-Z cross section elevation view of the partially rotating sheathed needle actuation device upon initiation of sheathing, and enlarged portion thereof in FIG. 11F. FIGS. 11A-F illustrate initial retraction following completed injection by the user, retracting the partially rotating sheathed needle actuation device 100 from injection site 500. Due to the freedom of the shield member 120 to move as described in FIG. 10 above and shown in FIG. 11A, the shield member 120 can remain in contact with injection site 500 and retract from sheath member 118 and sleeve member 112 (see e.g., FIG. 11E and described in FIG. 13 stage 194), while locking arms 156 of shield member 120 (see e.g., FIG. 11E) are disengaged from recesses 146 (see e.g., FIG. 11E) of sheath member 118 and slide over the exterior cylindrical surface thereof. As illustrated, disengaging sheath member 118 from the sub-assembly of sheath member 118, biaser 116 and shield member 120, causes armed biaser 116 to bias sheath member abutting rim 142 (see e.g., FIG. 11F) 118 away from shield member 120 (see e.g., FIG. 11A).

As illustrated, the combination of shelves 148 of sheath member 118 (see e.g., FIG. 11D) and the front portions 165 of rails 154 (see e.g., FIG. 11E, 11F) of shield member 120 can be released from intermediate portion 135_{I} of sleeve member 112, so that front facet 150 of shelves 148 (see e.g., FIG. 11D) contiguously slidably translate distally along the longitudinal face of abutment 138 of sleeve member 112 (see e.g., FIG. 9B)

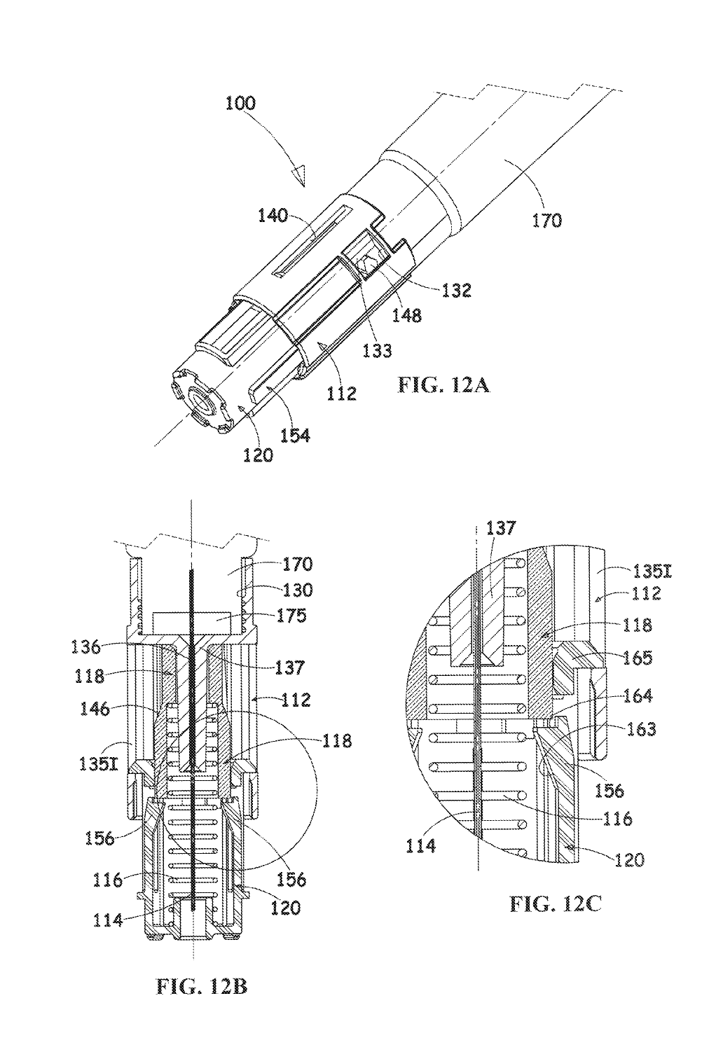

FIGS. 12A-C shows a cross section of the partially rotating sheathed needle actuation device 100 upon completion of needle sheathing (12A) with enlarged section G (12B). As illustrated, at complete deactivation configuration after the user fully removed partially rotating sheathed needle actuation device 100 away from the injection site 500, in which resilient locking arms 156 of shield member 120 have surpassed the anterior portion of sheath member 118 locking shield member 120 at the proximal end of sleeve member 112 (see e.g., FIG. 12C). The result is that the distal end of resilient arms 156 can relax forcing ledge 164 (see e.g., FIG. 12C) to abut flush portion 143 of sheath member 118 proximal end (see e.g., FIG. 4A), whereby shield member 120 can then retracted fully—covering the proximal end of needle cannula 114, and preventing shield member 120 from sliding distally.

The thickness of resilient locking arms 156 can be adapted to provide the required force distance profile of shield member 120, by, for example, controlling the friction exerted on shieth member 118. Other factors that can be used to adjust the profile, can be, inter-alia:

i. the slope angle of beveled proximal end of sheath member 118; and/or

ii. the depth of recessed portion 146; and/or

iii. angle of radial beveled facet 133 of sleeve member 112; and/or

iv. angle of first slanted facet 158, secnd axially parallel facet 159, and second slanted facet 160 of rail 156 of shield member 120; and/or

v. size of shelves 148 and size and angles of front facet 150, upper dovetail facet 151, lower dovetail facet 149, plane back facet 152 and chamfered facet 153; and/or

vi. size and strength of biaser 116 and/or a combination comprising one or more of the foregoing.

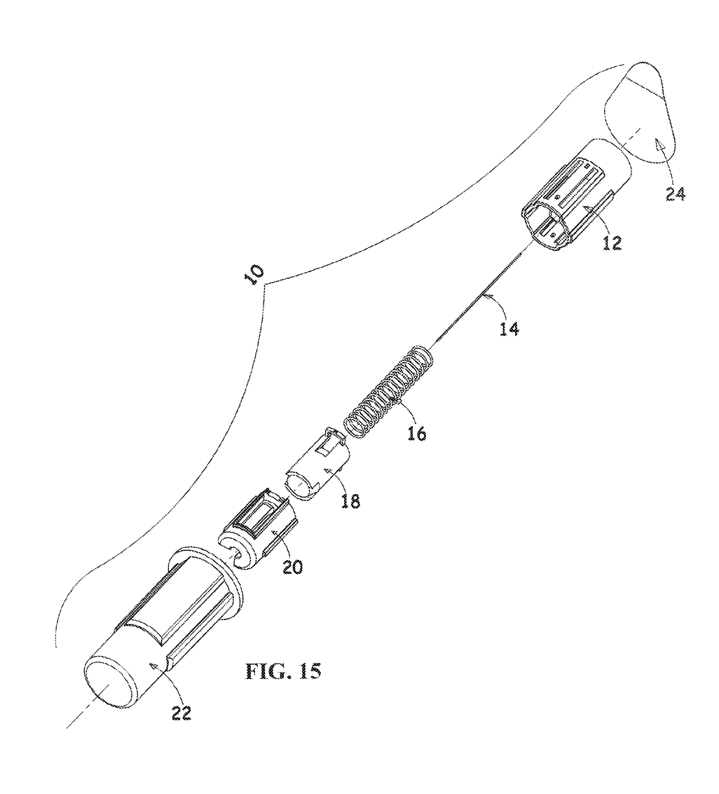

Turning now to FIG. 13, illustrating a comparison between the sheathed needle actuation devices 100, 10 described herein (Solid line) and currently available sheathed needle actuation devices (dashed line). In the graph, in which the magnitude of force in Newtons (N) acting shield member 120, 20 (see e.g., FIG. 15), is plotted as a function of the distance in millimeters (mm) of advancement and retreat of the shield member 120, (see inset e.g., device 100) 20 relative to sleeve member 112, 12 (see e.g., FIG. 15) first distally, then proximally, during the actuation of the sheathed needle actuation devices 100, 10. During intial phase 186 the initial actuation of the sheathed needle actuation devices 100, 10 takes place, i.e. the minor rotation in a clockwise direction (see e.g., FIG. 8), resulting in peak force 198 (section C-C), representing the urging of needle cannula 114, 14 (see e.g., FIG. 15). The sharp decrease in the force, during phase 188, represents the smooth linear motion and translation of the engaged shield members 120, 20 and sheath members 118, 18 (see e.g., FIGS. 10B, 15) relative to sleeve members 112, 12 respectively. During phase 190 (section D-D) where needle cannulas 114, 14 penetrated the injection site, the engaged shield members 120, 20 and sheath members 118, 18 slidably translate distally within sleeve members 112, 12. The rapid increase in the force during phase 192, represents the completion of injection and the subsequent initiation of exertion force by biaser 116, 16 (see e.g., FIGS. 12B, 15). During retraction stage 194, decrease in force is shown, representing the proximal advancement of shield member 120, 20 whereas the relatively more moderate to flat changes in the force during phase 196, represents the proximal advancement of shield 120, 20 while sliding over the exterior cylindrical surface of sheath member 118, 18. To actuate the device, the user operating the sheathed needle actuation devices 100, 10, a user has to exert a force exceeding the threshold value of phase 186. Therefore, the abrupt decrease in the force, during phase 188, facilitates a complete and continuous advancement of shield member 120 into the sleeve member 112 during stage 190, and hence an automatic needle insertion is achieved, without the recoil that can result in the currently available devices.

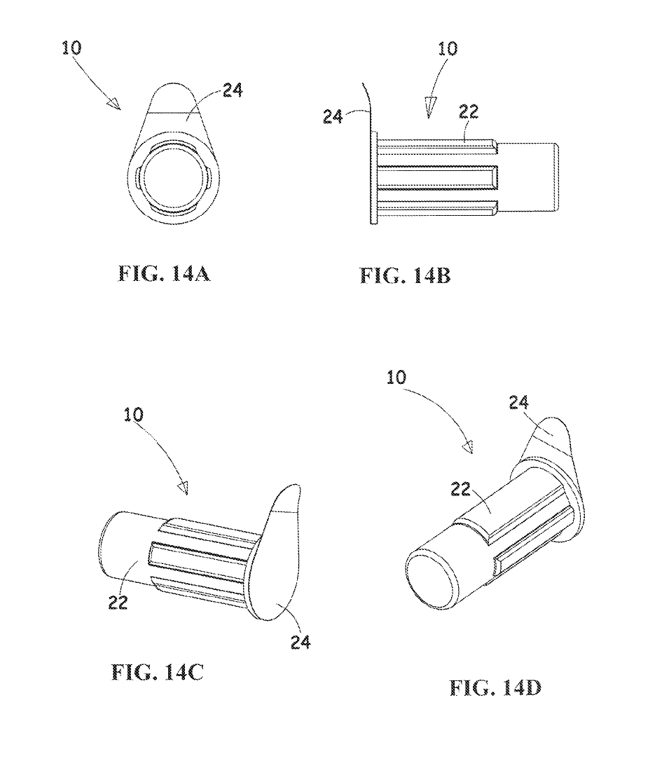

Turning now to FIG. 14, illustrating top (14A) plan view, side (14B) elevation view, bottom perspective view (14C), and top perspective view (14D) of the linear motion sheathed needle actuation device 10. As illustrated, linear motion sheathed needle actuation device 10 can be configured to provide a predetermined force-distance profile during injection, and can be enclosed in housing 22, having an open distal end sealed with a peelable cover tab 24.

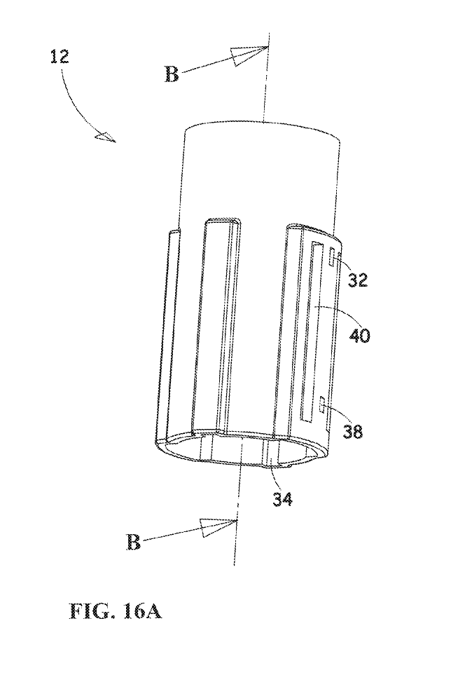

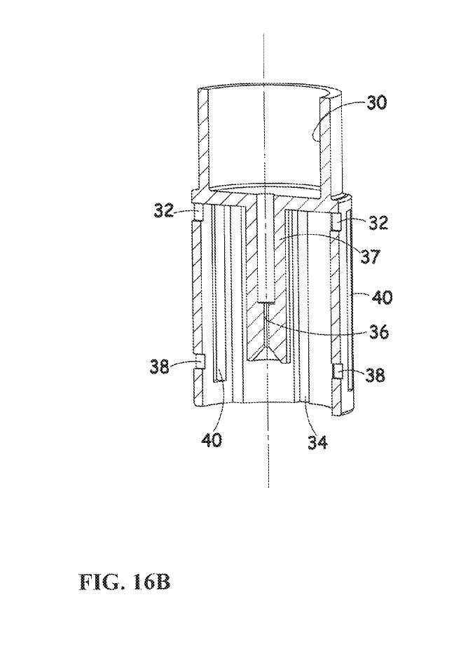

Moving to FIGS. 15-18, showing in FIG. 15 an exploded isometric view of linear motion sheathed needle actuation device 10 as shown in FIGS. 14A-14D. Device 10 can comprise housing member 22 having a longitudinal axis, proximal end and a distal end. Sleeve member 12 can be adapted to receive and engage a proximal end of a body (for example, an autoinjection pen) comprising an injectable compound (not shown). Sleeve 12 can have a longitudinal axis, a proximal end and a distal end and define central axial flanged column 37 (See e.g., FIG. 16), which can be configured to receive and engage needle cannula 14 having a proximal end and a distal end. Needle cannula 14 can have a proximal end and a distal end, and be operably coupled to sleeve member 12. Also shown is shield member 20 having a longitudinal axis, a distal end that can be slidably coupled to central flanged column 37 (see e.g., FIG. 16) of sleeve member 12 and a proximal end defining a central aperture accommodating the proximal end of needle cannula 14. FIG. 15 further shows sheath member 18 having an open distal end and open proximal end. Sheath member 18 can be moveably slidably coupled to shield member 20 and be configured to move between a first position surrounding needle cannula 14 and a second position exposing needle cannula 14. Also illustrated in FIG. 15 is biaser 16 operably coupled to shield member 20 for biasing needle cannula 14 toward proximal end, wherein the assembly is configured to provide a predetermined profile (see e.g., FIG. 13, 24) of force on shield member 20 as a function of distance traveled by shield member 20 during injection.

Turning now to FIG. 16, illustrating isometric perspective view in FIG. 16A and X-Z cross section B-B of sleeve member 12 of FIG. 15 in FIG. 16B of linear motion sheathed needle actuation device 10. As shown in FIG. 16B, sleeve member 12 can comprise an injector engaging portion 30 disposed at the distal end of sleeve member 12, with flanged needle column 37, having a needle bore 36 configured to receive and engage needle cannula 14. Sleeve member 12 can also comprise at least a pair of radially disposed distal openings 32 (see e.g., FIGS. 16A, 16B), wherein distal openings 32 can be disposed toward sleeve member's 12 distal end above the flanged portion of flanged needle column 37. Sleeve member 12 can further comprise at least pair of shield member guiding grooves 34. At least a pair of radially disposed proximal openings 38 can be defined, wherein each of proximal openings 38 can be disposed toward sleeve member's 12 proximal end, each proximal opening 38 axially aligned with a corresponding distal opening 32. Sleeve member 12 can further comprise at least a pair of shield member guiding slots 40.

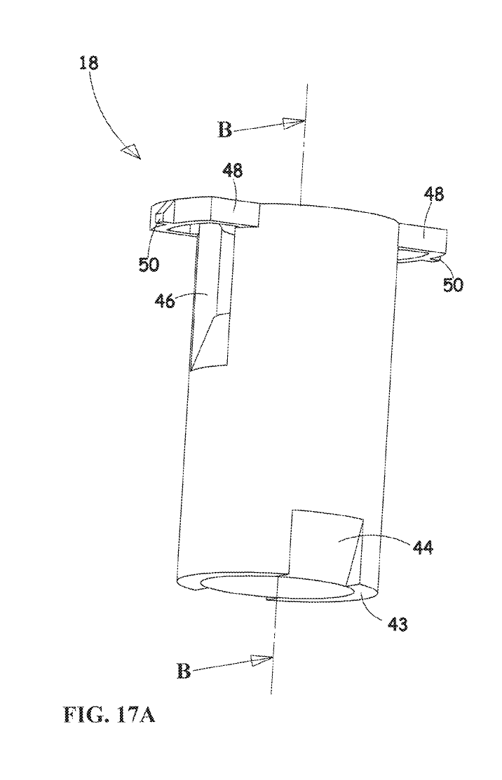

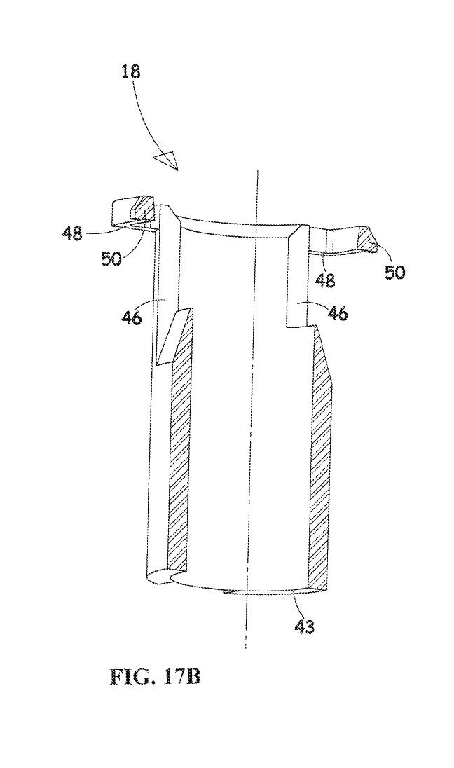

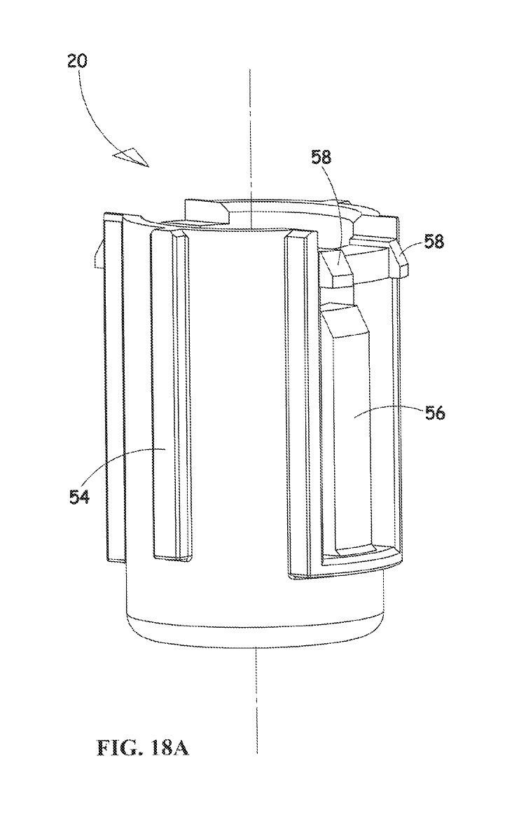

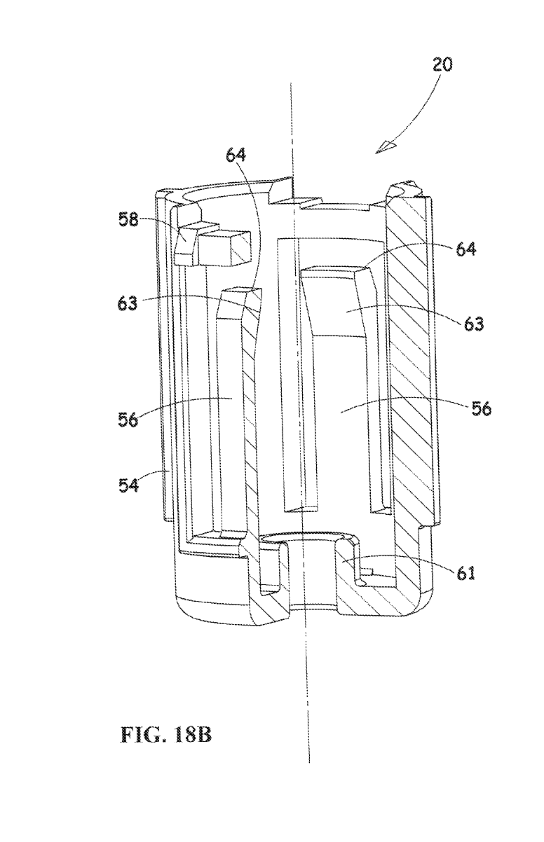

FIG. 17A illustrates an isometric perspective view of sheath member 18 of linear motion sheathed needle actuation device 10. As shown (both in FIGS. 17A, 17B), sheath member 18 can comprise beveled proximal end 44, separatd by flush portion 43; recessed portion 46 configured to receive and engage at least one resilient locking arm 56 (see e.g., FIG. 18) and at least a pair of radially disposed distal brackets 48, each having a centrally disposed protuberance 50.

Turning now to FIG. 18B, illustrating cutaway view of shield member 20 of linear motion sheathed needle actuation device 10. As illustrated, in FIG. 18A, an isometric perspective view, shield member 20 can comprise at least a pair of guiding rails 54 configured to be received in sleeve member 12 guiding slots 40. Also shown, are at least a pair of resilient locking arms 56, having distal end terminating in a sloped expansion 63 (See e.g., FIG. 18B) with ledge 64, sloped expansion 63 configured to engage sheath member 18 recessed portion 46. Shield member 20 can further comprise at least a pair of guiding perturberances 58 and a concentric flanged ring member 61, configured to engage biaser 16. Ring member 61 can be disposed at the open proximal end of shield member 20.

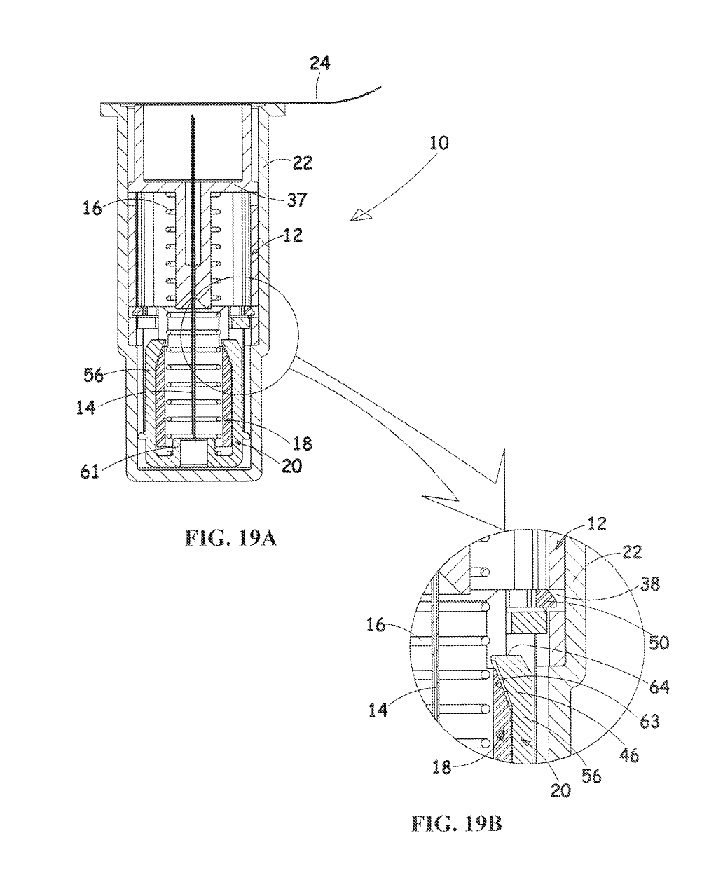

Turning now to FIG. 19, illustrating a cross section of the linear motion sheathed needle actuation device 10 in stowed initial position (19A) and enlarged section H (19B). As illustrated, components of linear motion sheathed needle actuation device 10 are enclosed within housing 22 hermetically sealed by detachable cover reed or tab 24. Perturberances 50 of sheath member 18 are disposed in proximal opening 38 of sleeve member 12, in such a manner that the chamfered faces on perturberances 50 facing the edges formed by proximal opening 38, locking resilient arms 56 of shield member 20 are disposed in clearances 46 of sheath member, and can be aligned with flush portion 44 of sheath member 18 proximal end.

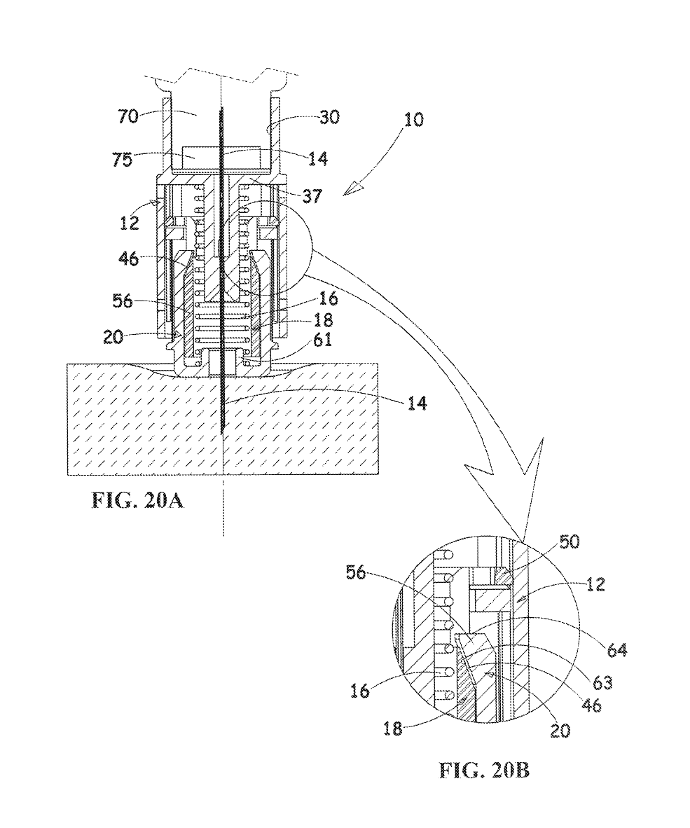

Turning now to FIG. 20, showing a cross section of the linear motionsheathed needle actuation device 10 upon partial actuation (20A) and enlarged section I (20B). As illustrated, in a partially activated configuration, housing 22 and cover reed 24 can be removed and injector engaging portion 30 of sleeve member 12 can be operably coupled to injector device 70 so that needle 14 penetrates into septum 75 thereof.

In order to achieve a partial activation of linear motion sheathed needle actuation device 10, the user presses shield member 20 against the injection site 500 (see e.g., FIG. 8A); thereby a force is exerted on shield member 20 and consequently on sheath member 18, urging the latter in direction of injector engaging portion 30 of sleeve member 12. Subsequently, perturberances 50 of sheath member 18 are released from proximal opening 38 of sleeve member 12 and slideably translated across the interior surface of sleeve member 12 while biaser 16 is being gradually compressed. In the initially activated configuration, distal end 63 of locking arms 56 of shield member 20 are disposed in clearances 46 of sheath member 18.

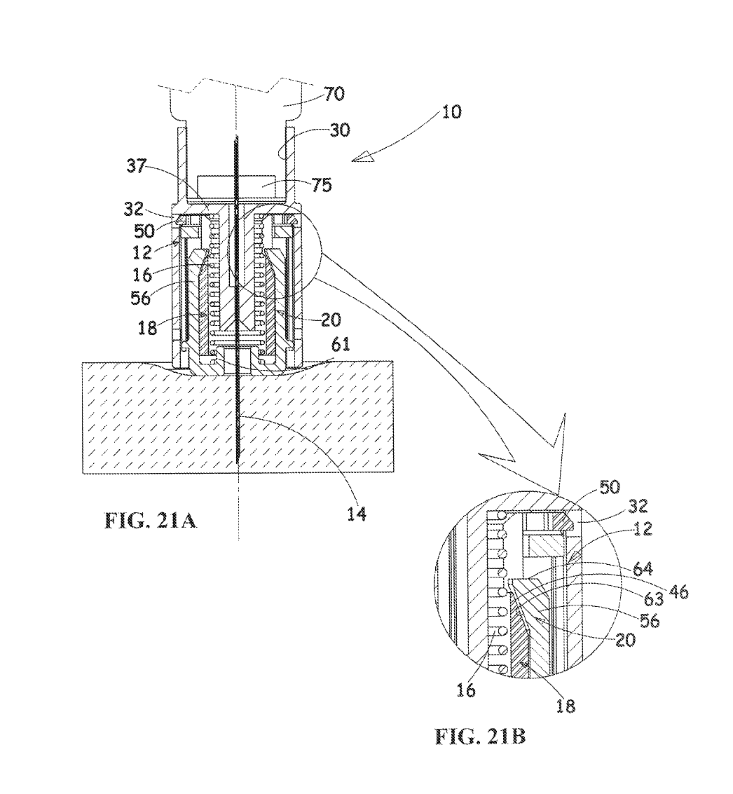

Turning now to FIG. 21, illustrating a cross section of linear motionsheathed needle actuation device 10 during injection (21A) and enlarged section J (21B). As illustrated, in a completely actuated configuration, perturberances 50 of sheath member 18 are disposed in distal opening 32, shield member 20 is retracted into sleeve member 12 and biaser 16 is compressed substantially to its greatest extent. While completely actuated, needle cannula 14 extends therefrom to an essentially maximal extent, the user typically performs an injection of an injectable contained in injector 70.

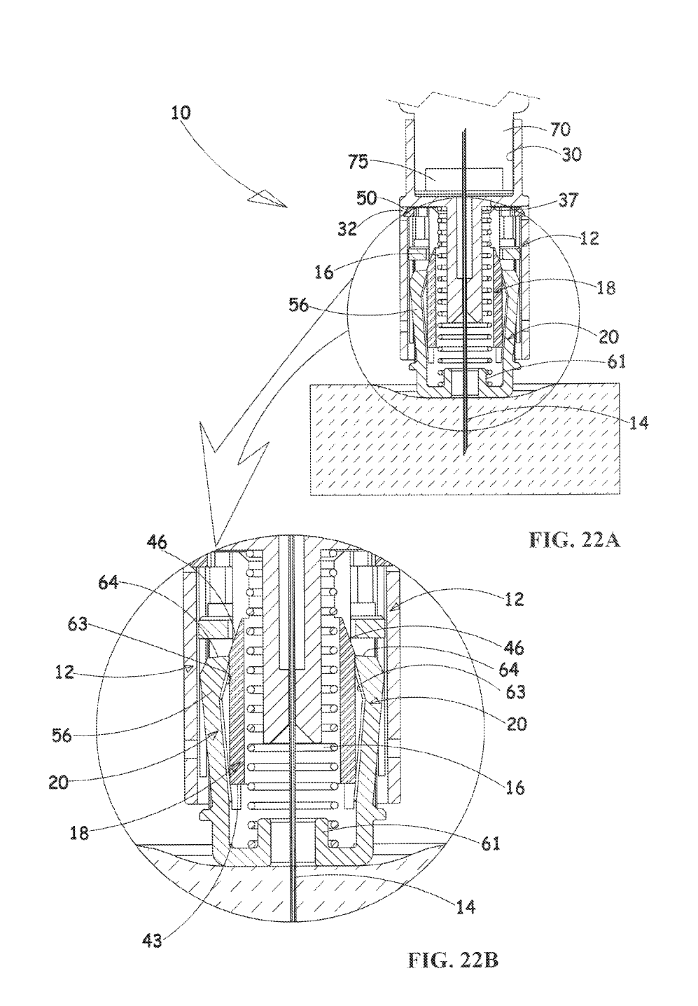

FIG. 22, shows a cross section of the linear motionsheathed needle actuation device upon completion of initial actuation (22A) and enlarged section K (22B). FIGS. 22A and 22B, at the end of the injection, upon the user receiving, for example, a visual confirmation of the end of the injection, the user will retract needle cannula 14 from the injection site, causing biaser 16 to urge shiled member proximally, while perturberances 50 of sheath member 18 with flat portion abuting the proximal end of distal opening 32, prevent sheath member 18 from moving proximally, causing sloped expansion 63 disposed on the distal end of resilient locking arms 56, to expand and slide or glide over recesses 46 of sheath member 18.

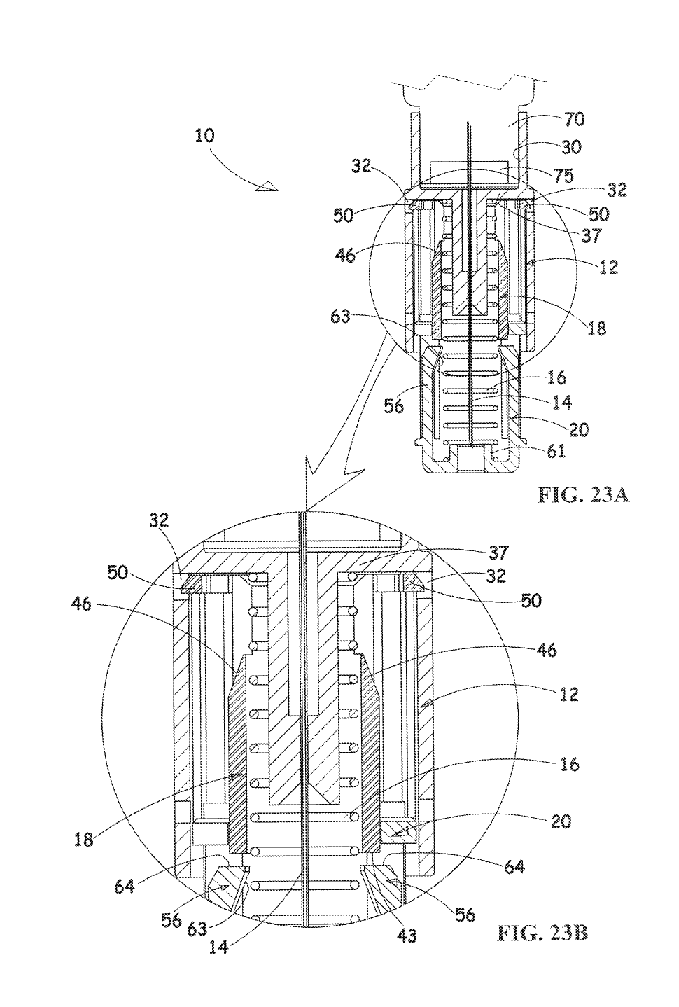

Finally, FIG. 23, shows a cross section of the linear motionsheathed needle actuation device upon completion of needle sheathing (23A) and enlarged section L (12B). As illustrated, once ledge 64 disposed at the distal end of resilient arms 56 of shield member 20 surpass beyond the distal end of sheath member 18, the resilient locking arms relax, and contract over the flush portion 43 of sheath member 18 distal end, causing shield member, now completely covering needle cannula 14, to lock in place and prevent shield member 20 from further movement distally.

The thickness of resilient locking arms 56 can be adapted to provide the required force distance profile of shield member 20, by, for example, controlling the friction exerted on shieth member 118. Other factors that can be used to adjust the profile, can be, inter-alia:

vii. proximal angle of recess portion 46 of sheath member 18;

viii. sloped expansion angle 63 of the distal end of resilient locking arm 56;

ix. biaser strength;

x. polymer used for producing the shield member;

xi. distal angle of porturberances 50;

xii. relative axial length ratio of shield member 20 to sheath member 18;

xiii. axial distance between aligned proximal opening 38 and distal opening 32;

and a combination comprising one or more of the foregoing. As indicated previously, these and other factors can be used in certain embodiments with fine tuning the profile of all devices described herein.

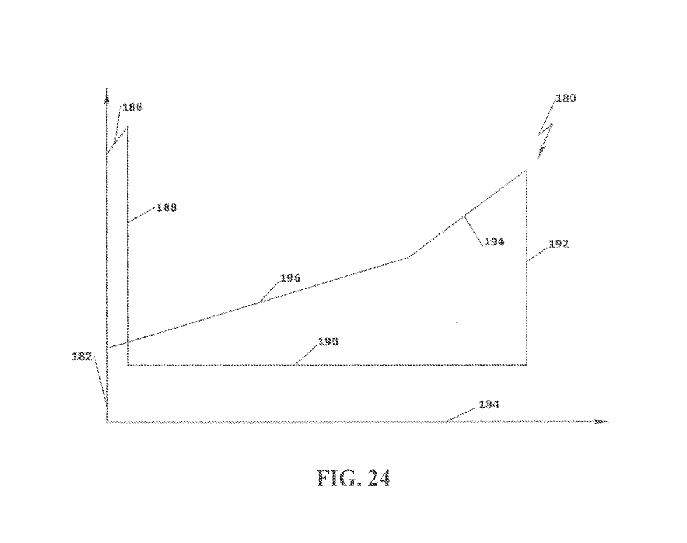

Turning now to FIG. 24, showing the force distance profile of the partially rotating embodiment of the sheathed needle actuation device 100. As illustrated in FIG. 24, the magnitude of force shield member 120 is exposed to, is represented on Y-axis and is plotted as a function of distance of movement of the shield member 120 relative the sleeve member 112. During the activation of partially rotating embodiment of the sheathed needle actuation device 100 as described herein; in phase 186 the initial activation of partially rotating embodiment of the sheathed needle actuation device 100 is performed, i.e. rotation in a clockwise direction. The sharp decrease in the force, during phase 188, represents the introduction of the coupled shelves 148 and front portions 165 of guiding rails 154 into intermediate portion 1351 of sleeve member 112. During phase 190 where needle cannula 114 penetrates the injection site, the coupled shelves 148 and front portions 165 of guiding rails 154 is in motion within intermediate portion 1351 of sleeve member 112, so facet 150 of shelves 148 are contiguously in slidable motion distally, along the longitudinal face of abutment 138. The sharp in the force, during phase 192 occurs when needle cannula 114 has fully penetrated the injection site, and represents the completion of activation of partially rotating embodiment of the sheathed needle actuation device 100 and the initiation of retraction force by the compressed biaser 116. The completion of activation of partially rotating embodiment of the sheathed needle actuation device 100 can be achieved by the separation of shelves 148 from guiding rails 154 and advancement thereof into quadrilateral openings 132, i.e. the rotation of sheath member 118 in counter-clockwise direction relatively to sleeve member 112 and shield member 120, causing locking arms 156 to be aligned with flush portion 143 of the proximal end of sheath member 118. The relatively less moderate decrease in the force, during phase 194 occurs when the user starts to remove shield member 120 from the injection site, illustrates the advancement of shield member 120 proximally while locking arms 156 of shield member 120 are being displaced from clearances 146 in sheath member 118. The relatively more moderate decrease in the force, observed during phase 196, occurs due to the proximal advancement of shield member 120 while sloped expansion 163 disposed on the distal end of locking arms 156 slide or glide over the exterior surface of sheath member 118.

While in the foregoing specification the surgical cranial drape, microelectrodes for mapping brain of a subject and their methods of use have been described in relation to certain preferred embodiments, and many details are set forth for purpose of illustration, it will be apparent to those skilled in the art that the disclosure of the surgical cranial drape, microelectrodes for mapping brain of a subject and their methods of use are susceptible to additional embodiments and that certain of the details described in this specification and as are more fully delineated in the following claims can be varied considerably without departing from the basic principles of this invention.

1. A safety needle shield assembly comprising:
a. a sleeve member adapted to receive and engage a proximal end of a body comprising an injectable compound, having a longitudinal axis, a proximal end and a distal end;
b. a needle cannula having a proximal end and a distal end, operably coupled to the sleeve member;
c. a shield member having a longitudinal axis, a distal end coupled to a sheath member and a proximal end defining a central aperture accommodating the proximal end of the needle cannula;
d. the sheath member having an open distal end and open proximal end, the sheath member being moveably slidably coupled to the shield member, the shield member configured to move between a first position surrounding the needle cannula and a second position exposing the needle cannula; and
e. a biaser operably coupled to the needle shield for biasing the shield member toward proximal end, wherein the assembly is configured to provide a predetermined profile of force on the shield as a function of distance traveled by the shield during the movement of the shield member relative to the sleeve member.

2. The assembly of claim 1, wherein the shield is configured to be exposed to a force of between about 2.5 N and about 3.5 N within about 0.2 mm and about 1.2 mm.

3. The assembly of claim 1, wherein following an initial peak force, the shield member is exposed to a lower, substantially constant force.

4. The assembly of claim 3, wherein following an initiation of movement at the peak force, between about 2.0 mm and about 9.4 mm, the shield is configured to be exposed to an increase in force of between about 0.2 N and about −0.4 N.

5. The assembly of claim 1, wherein following initiation of movement, between about 9.0 mm and about 11 mm, the shield is configured to be exposed to a force of between about 2.8 N and about 3.8 N.

6. The assembly of claim 3, wherein the shield member is configured to be exposed to a peak force of no more than 3.5 N, associated with the actuation of needle penetration at an injection site.

7. The assembly of claim 3, wherein the shield movement is associated with injection of the injectable compound.

8. The assembly of claim 4, wherein the shield is configured to be exposed to a peak force of no more than 3.5 N, associated with the actuation of the sheath by the biaser.

9. The assembly of claim 1, wherein the sheath member being moveably slidably coupled to the shield member between a first position surrounding the needle cannula and a second position exposing the needle cannula, is adapted to partially rotate upon movement between the first position surrounding the needle cannula and the second position exposing the needle cannula.

10. The assembly of claim 1, wherein the sleeve member further comprises:
a. an injector engaging portion disposed on the distal end of the sleeve;
b. a flanged needle column, having a needle bore, configured to receive and engage the needle canula;
c. a central, coaxial flanged column, configured to engage the cannula;
d. at least a pair of quadrilateral distal opening disposed radially above the flanged portion of the needle column, each quadrilateral opening having a pair of parallel axial facets and a pair of parallel radial facets, wherein the radial facets disposed closer to the distal end of the sleeve are beveled;
e. at least a pair of axial grooves, each having an anterior channel portion, an intermediate portion and a posterior portion, wherein an abutment extends along the entire length of the intermediate portion; and
f. at least a pair of shield member guiding slots.

11. The assembly of claim 9, wherein the shield member comprises
a. at least a pair of rails configured to be received in the sleeve member guiding slots, wherein each of the rails further defines a graded recess formed by a first axial facet, a first slanted facet, a second axial facet and a second slanted facet;
b. a pair of resilient locking arms; and
c. a concentric flanged ring member, configured to couple to the biaser

12. The assembly of claim 1, wherein the sheath member comprises:
a. a proximal end having a beveled portion and a flush portion;
b. a distally positioned recessed portion; and
c. at least a pair of shelves, each shelf comprising a front facet, an upper dovetail facet, a lower dovetail facet, a plane back facet and a chamfered facet.

13. The assembly of claim 1, wherein, in the stowed position, the biaser is compressed between the shield member and the sheath member.

14. The assembly of claim 7, wherein the shield member, the sheath and the biaser are configured to move together.

15. The assembly of claim 1, wherein the sleeve member further comprises:
a. an injector engaging portion disposed on the distal end of the sleeve;
b. a flanged needle column, having a needle bore configured to receive and engage the needle cannula;
c. at least a pair of radially disposed distal openings, wherein the openings are disposed toward the sleeve member's distal end above the flanged portion of the needle column;
d. at least a pair of shield member guiding grooves;
e. at least a pair of radially disposed proximal openings, wherein each of the openings are disposed toward the sleeve member's proximal end, each proximal opening axially aligned with a corresponding distal opening; and
f. at least a pair of shield member guiding slots.

16. The assembly of claim 15, wherein the shield member comprises
a. at least a pair of guiding rails configured to be received in the sleeve member guiding slots;
b. at least a pair of resilient locking arms, configured to engage the sheath member;
c. at least a pair of guiding projections; and
d. a concentric flanged ring member, configured to receive and engage the biaser.

17. The assembly of claim 16, wherein the sheath member comprises:
a. a proximal end having a beveled portion and a flush portion;
b. a distally disposed recessed portion configured to receive and engage at least one resilient locking arm of the shield member; and
c. at least a pair of radially disposed distal brackets having a centrally disposed protuberance.

18. The assembly of claim 15, wherein the sheath member being moveably slidably coupled to the shield member between a first position surrounding the needle cannula and a second position exposing the needle cannula, is adapted for linear movement between the first position surrounding the needle cannula and the second position exposing the needle cannula.

19. The assembly of claim 15, wherein the biaser is adapted to be compressed upon coupling to the injector.

20. A safety needle shield assembly comprising:
a. a sleeve member adapted to receive and engage a proximal end of a body comprising an injectable compound, having a longitudinal axis, a proximal end and a distal end;
b. a needle cannula having a proximal end and a distal end, operably coupled to the sleeve member;
c. a shield member having a longitudinal axis, a distal end coupled to a sheath member and a proximal end defining a central aperture accommodating the proximal end of the needle cannula;
d. the sheath member having an open distal end and open proximal end, the sheath member being moveably slidably coupled to the shield member, the shield member configured to move between a first position surrounding the needle cannula and a second position exposing the needle cannula; and
e. a biaser operably coupled to the needle shield for biasing the shield member toward proximal end, wherein the shield member, sheath member and biaser are all configured to act as a single component in the second position exposing the needle cannula.
